SEQUENCE LISTING (30 sequences)

SEQ ID NO: 1 - DNA, 4588 bp, Human
gcggccgccaagagagatcacacccccagccgaccctgccagcgagcgagcccgaccccaggcgtccatggagcgtcgcctccgcccggtccctgccccgacccccgcctgcggcgcggctcctgccttgaccaggacttgggactttgcgaaaggatcgcggggcccggagaggtaaccgccgcgcctcccggagaggtgttggagagcacaatggctgaacaagtccttcctcaggctttgtatttgagcaatatgcggaaagctgtgaagatacgggagagaactccagaagacatttttaaacctactaatgggatcattcatcattttaaaaccatgcaccgatacacactggaaatgttcagaacttgccagttttgtcctcagtttcgggagatcatccacaaagccctcatcgacagaaacatccaggccaccctggaaagccagaagaaactcaactggtgtcgagaagtccggaagcttgtggcgctgaaaacgaacggtgacggcaattgcctcatgcatgccacttctcagtacatgtggggcgttcaggacacagacttggtactgaggaaggcgctgttcagcacgctcaaggaaacagacacacgcaactttaaattccgctggcaactggagtctctcaaatctcaggaatttgttgaaacggggctttgctatgatactcggaactggaatgatgaatgggacaatcttatcaaaatggcttccacagacacacccatggcccgaagtggacttcagtacaactcactggaagaaatacacatatttgtcctttgcaacatcctcagaaggccaatcattgtcatttcagacaaaatgctaagaagtttggaatcaggttccaatttcgcccctttgaaagtgggtggaatttacttgcctctccactggcctgcccaggaatgctacagataccccattgttctcggctatgacagccatcattttgtacccttggtgaccctgaaggacagtgggcctgaaatccgagctgttccacttgttaacagagaccggggaagatttgaagacttaaaagttcactttttgacagatcctgaaaatgagatgaaggagaagctcttaaaagagtacttaatggtgatagaaatccccgtccaaggctgggaccatggcacaactcatctcatcaatgccgcaaagttggatgaagctaacttaccaaaagaaatcaatctggtagatgattactttgaacttgttcagcatgagtacaagaaatggcaggaaaacagcgagcaggggaggagagaggggcacgcccagaatcccatggaaccttccgtgccccagctttctctcatggatgtaaaatgtgaaacgcccaactgccccttcttcatgtctgtgaacacccagcctttatgccatgagtgctcagagaggcggcaaaagaatcaaaacaaactcccaaagctgaactccaagccgggccctgaggggctccctggcatggcgctcggggcctctcggggagaagcctatgagcccttggcgtggaaccctgaggagtccactggggggcctcattcggccccaccgacagcacccagcccttttctgttcagtgagaccactgccatgaagtgcaggagccccggctgccccttcacactgaatgtgcagcacaacggattttgtgaacgttgccacaacgcccggcaacttcacgccagccacgccccagaccacacaaggcacttggatcccgggaagtgccaagcctgcctccaggatgttaccaggacatttaatgggatctgcagtacttgcttcaaaaggactacagcagaggcctcctccagcctcagcaccagcctccctccttcctgtcaccagcgttccaagtcagatccctcgcggctcgtccggagcccctccccgcattcttgccacagagctggaaacgacgcccctgctggctgcctgtctcaagctgcacggactcctggggacaggacggggacgagcaagtgcagaaaagccggctgcgtgtattttgggactccagaaaacaagggcttttgcacactgtgtttcatcgagtacagagaaaacaaacattttgctgctgcctcagggaaagtcagtcccacagcgtccaggttccagaacaccattccgtgcctggggagggaatgcggcacccttggaagcaccatgtttgaaggatactgccagaagtgtttcattgaagctcagaatcagagatttcatgaggccaaaaggacagaagagcaactgagatcgagccagcgcagagatgtgcctcgaaccacacaaagcacctcaaggcccaagtgcgcccgggcctcctgcaagaacatcctggcctgccgcagcgaggagctctgcatggagtgtcagcatcccaaccagaggatgggccctggggcccaccggggtgagcctgcccccgaagacccccccaagcagcgttgccgggcccccgcctgtgatcattttggcaatgccaagtgcaacggctactgcaacgaatgctttcagttcaagcagatgtatggctaaccggaaacaggtgggtcacctcctgcaagaagtggggcctcgagctgtcagtcatcatggtgctatcctctgaacccctcagctgccactgcaacagtgggcttaagggtgtctgagcaggagaggaaagataagctcttcgtggtgcccacgatgctcaggtttggtaacccgggagtgttcccaggtggccttagaaagcaaagcttgtaactggcaagggatgatgtcagattcagcccaaggttcctcctctcctaccaagcaggaggccaggaacttctttggacttggaaggtgtgcggggactggccgaggcccctgcaccctgcgcatcaggactgcttcatcgtcttggctgagaaagggaaaagacacacaagtcgcgtgggttggagaagccagagccattccacctcccctcccccagcatctctcagagatgtgaagccagatcctcatggcagcgaggccctctgcaagaagctcaaggaagctcagggaaaatggacgtattcagagagtgtttgtagttcatggtttttccctacctgcccggttcctttcctgaggacccggcagaaatgcagaaccatccatggactgtgattctgaggctgctgagactgaacatgttcacattgacagaaaaacaagctgctctttataatatgcaccttttaaaaaattagaatattttactgggaagacgtgtaactctttgggttattactgtctttacttctaaagaagttagcttgaactgaggagtaaaagtgtgtacatatataatatacccttacattatgtatgagggatttttttaaattatattgaaatgctgccctagaagtacaataggaaggctaaataataataacctgttttctggttgttgttggggcatgagcttgtgtatacactgcttgcataaactcaaccagctgcctttttaaagggagctctagtcctttttgtgtaattcactttatttattttattacaaacttcaagattatttaagtgaagatatttcttcagctctggggaaaatgccacagtgttctcctgagagaacatccttgctttgagtcaggctgtgggcaagttcctgaccacagggagtaaattggcctctttgatacacttttgcttgcctccccaggaaagaaggaattgcatccaaggtatacatacatattcatcgatgtttcgtgcttctccttatgaaactccagctatgtaataaaaaactatactctgtgttctgttaatgcctctgagtgtcctacctccttggagatgagatagggaaggagcagggatgagactggcaatggtcacagggaaagatgtggccttttgtgatggttttattttctgttaacactgtgtcctgggggggctgggaagtcccctgcatcccatggtaccctggtattgggacagcaaaagccagtaaccatgagtatgaggaaatctctttctgttgctggcttacagtttctctgtgtgctttgtggttgctgtcatatttgctctagaagaaaaaaaaaaaaaggaggggaaatgcattttccccagagataaaggctgccattttgggggtctgtacttatggcctgaaaatatttgtgatccataactctacacagcctttactcatactattaggcacactttccccttagagccccctaagtttttcccagacgaatctttataatttctttccaaagataccaaataaacttcagtgttttcatctaattctcttaaagttgatatcttaatattttgtgttgatcattatttccattcttaatgtgaaaaaaagtaattatttatacttattataaaaagtatttgaaatttgcacatttaattgtccctaatagaaagccacctattctttgttggatttcttcaagtttttctaaataaatgtaacttttcacaagagtcaacattaaaaaataaattatttaaaaaaaaaaaaaaa

SEQ ID NO: 2 - protein, 790 aa, Human
MAEQVLPQALYLSNMRKAVKIRERTPEDIFKPTNGIIHHFKTMHRYTLEMFRTCQFCPQFREIIHKALIDRNIQATLESQKKLNWCREVRKLVALKTNGDGNCLMHATSQYMWGVQDTDLVLRKALFSTLKETDTRNFKFRWQLESLKSQEFVETGLCYDTRNWNDEWDNLIKMASTDTPMARSGLQYNSLEEIHIFVLCNILRRPIIVISDKMLRSLESGSNFAPLKVGGIYLPLHWPAQECYRYPIVLGYDSHHFVPLVTLKDSGPEIRAVPLVNRDRGRFEDLKVHFLTDPENEMKEKLLKEYLMVIEIPVQGWDHGTTHLINAAKLDEANLPKEINLVDDYFELVQHEYKKWQENSEQGRREGHAQNPMEPSVPQLSLMDVKCETPNCPFFMSVNTQPLCHECSERRQKNQNKLPKLNSKPGPEGLPGMALGASRGEAYEPLAWNPEESTGGPHSAPPTAPSPFLFSETTAMKCRSPGCPFTLNVQHNGFCERCHNARQLHASHAPDHTRHLDPGKCQACLQDVTRTFNGICSTCFKRTTAEASSSLSTSLPPSCHQRSKSDPSRLVRSPSPHSCHRAGNDAPAGCLSQAARTPGDRTGTSKCRKAGCVYFGTPENKGFCTLCFIEYRENKHFAAASGKVSPTASRFQNTIPCLGRECGTLGSTMFEGYCQKCFIEAQNQRFHEAKRTEEQLRSSQRRDVPRTTQSTSRPKCARASCKNILACRSEELCMECQHPNQRMGPGAHRGEPAPEDPPKQRCRAPACDHFGNAKCNGYCNECFQFKQMYG

SEQ ID NO: 3 - DNA, 1224 bp, Human
  misc_feature 36, 91, 645, 655, 660, 671, 672: n = A,T,C or G
tcgggatcgatctggagctccgggaatttccctggnccgggactccgggctttccagccccaaccatgcataaaaggggttcgccgttctnggagagccacagagcccgggccacaggcagctccttgccagctcttcctctcctctcacagccgccagacccgcctgctgagcccccatggcccgcgctgctctctccgccgcccccagcaatccccggctcctgcgagtggcgctgctgctcctgctcctggtagccgctggccggcgcgcagcaggagcgcccctggccactgaactgcgctgccagtgcttgcagaccctgcagggaattcacctcaagaacatccaaagtgtgaaggtgaagtcccccggaccccactgcgcccaaaccgaagtcatagccacactcaagaatgggcagaaagcttgtctcaaccccgcatcgcccatggttaagaaaatcatcgaaaagatgctgaaaaatggcaaatccaactgaccagaaggaaggaggaagcttattggtggctgttcctgaaggaggccctgcccttacaggaacagaagaggaaagagagacacagctgcagaggccacctgggattgcgcctaatgtgtttgagcatcacttaggagaaggcnccgattaatnaattnattaatttatnnattggttggttttagaagattctatgttaatattttatgtgtaaaataaggttatgattgaatctacttgcacactctcccattatatttattgtttattttaggtcaaacccaagttagttcaatcctgattcatatttaatttgaagatagaaggtttgcagatattctctagtcatttgttaatatttcttcgtgatgacatatcacatgtcagccactgtgatagaggctgaggaatccaagaaaatggccagtaagatcaatgtgacggcagggaaatgtatgtgtgtctattttgtaactgtaaagatgaatgtcagttgttatttattgaaatgatttcacagtgtgtggtcaacatttctcatgttgaagctttaagaactaaaatgttctaaatatcccttggacattttatgtctttcttgtaaggcatactgccttgtttaatgttaattatgcagtgtttccctctgtgttagagcagagaggtttcgatatttattgatgttttcacaaagaacaggaaaataaaatatttaaaaatat

SEQ ID NO: 4 - protein, 107 aa, Human
MARAALSAAPSNPRLLRVALLLLLLVAAGRRAAGAPLATELRCQCLQTLQGIHLKNIQSVKVKSPGPHCAQTEVIATLKNGQKACLNPASPMVKKIIEKMLKNGKSN

SEQ ID NO: 5 - DNA, 1708 bp, Human
cgcagctctgtgtgaaggtgcagttttgccaaggagtgctaaagaacttagatgtcagtgcataaagacatactccaaactttcagagacagcagagcacacaagcttctaggacaagagccaggaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcatggtaccatgaaatatccagaacatacttatatgtaaagtattatttatttgaatctacaaaaaacaacaaataatttttaaatataaggattttcctagatattgcacgggagaatatacaaatagcaaaattgaggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagatatacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttactgtttctgattgtatggaaatataaaagtaaatatgaaacatttaaaatataatttgttgt

SEQ ID NO: 6 - protein, 99 aa, Human
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 7 - DNA, 1385 bp, Human
gccaaccattccaagtcaggggctcccaacaaatgatagaccaggcttccctgtaccagtattctccacagaaccagcatgtagagcagcagccacactacacccacaaaccaactctggaatacagtccttttcccatacctccccagtcccccgcttatgaaccaaacctctttgatggtccagaatcacagttttgcccaaaccaaagcttagtttcccttcttggtgatcaaagggaatctgagaatattgctaatcccatgcagacttcctccagtgttcagcagcaaaatgatgctcacttgcacagcttcagcatgatgcccagcagcgcctgtgaggccatggtggggcacgagatggcctctgactcttcaaacacttcactgccattctcaaacatgggaaatccaatgaacaccacacagttagggaaatcactttttcagtggcaggtggagcaggaagaaagcaaattggcaaatatttcccaagaccagtttctttcaaaggatgcagatggtgacacgttccttcatattgctgttgcccaagggagaagggcactttcctatgttcttgcaagaaagatgaatgcacttcacatgctggatattaaagagcacaatggacagagtgcctttcaggtggcagtggctgccaatcagcatctcattgtgcaggatctggtgaacatcggggcacaggtgaacaccacagactgctggggaagaacacctctgcatgtgtgtgctgagaagggccactcccaggtgcttcaggcgattcagaagggagcagtgggaagtaatcagtttgtggatcttgaggcaactaactatgatggcctgactccccttcactgtgcagtcatagcccacaatgctgtggtccatgaactccagagaaatcaacagcctcattcacctgaagttcaggagcttttactgaagaataagagtctggttgataccattaagtgcctaattcaaatgggagcagcggtggaagcgaaggcttacaatggcaacactgccctccatgttgctgccagcttgcagtatcggttgacacaattagatgctgtccgcctgttgatgaggaagggagcagacccaagtactcggaacttggagaacgaacagccagtgcatttggttcccgatggccctgtgggagaacagatccgacgtatcctgaagggaaagtccattcagcagagagctccaccgtattagctccattagcttggagcctggctagcaacactcactgtcagttaggcagtcctgatgtatctgtacatagaccatttgccttatattggcaaatctaagttgtttctatgacacaaacatatttagttcactattatatacagt

SEQ ID NO: 8 - protein, 402 aa, Human
MIDQASLYQYSPQNQHVEQQPHYTHKPTLEYSPFPIPPQSPAYEPNLFDGPESQFCPNQSLVSLLGDQRESENIANPMQTSSSVQQQNDAHLHSFSMMPSSACEAMVGHEMASDSSNTSLPFSNMGNPMNTTQLGKSLFQWQVEQEESKLANISQDQFLSKDADGDTFLHIAVAQGRRALSYVLARKMNALHMLDIKEHNGQSAFQVAVAANQHLIVQDLVNIGAQVNTTDCWGRTPLHVCAEKGHSQVLQAIQKGAVGSNQFVDLEATNYDGLTPLHCAVIAHNAVVHELQRNQQPHSPEVQELLLKNKSLVDTIKCLIQMGAAVEAKAYNGNTALHVAASLQYRLTQLDAVRLLMRKGADPSTRNLENEQPVHLVPDGPVGEQIRRILKGKSIQQRAPPY

SEQ ID NO: 9 - DNA, 1057 bp, Human
gccgcagcacctcctcgccagctcttcctctcctctcacagccgccagacccgcctgctgagccccatggcccgcgctgctctctccgccgcccccagcaatccccggctcctgcgagtggcgctgctgctcctgctcctggtagccgctggccggcgcgcagcaggagcgtccgtggccactgaactgcgctgccagtgcttgcagaccctgcagggaattcaccccaagaacatccaaagtgtgaacgtgaagtcccccggaccccactgcgcccaaaccgaagtcatagccacactcaagaatgggcggaaagcttgcctcaatcctgcatcccccatagttaagaaaatcatcgaaaagatgctgaacagtgacaaatccaactgaccagaagggaggaggaagctcactggtggctgttcctgaaggaggccctgcccttataggaacagaagaggaaagagagacacagctgcagaggccacctggattgtgcctaatgtgtttgagcatcgcttaggagaagtcttctatttatttatttattcattagttttgaagattctatgttaatattttaggtgtaaaataattaagggtatgattaactctacctgcacactgtcctattatattcattctttttgaaatgtcaaccccaagttagttcaatctggattcatatttaatttgaaggtagaatgttttcaaatgttctccagtcattatgttaatatttctgaggagcctgcaacatgccagccactgtgatagaggctggcggatccaagcaaatggccaatgagatcattgtgaaggcaggggaatgtatgtgcacatctgttttgtaactgtttagatgaatgtcagttgttatttattgaaatgatttcacagtgtgtggtcaacatttctcatgttgaaactttaagaactaaaatgttctaaatatcccttggacattttatgtctttcttgtaaggcatactgccttgtttaatggtagttttacagtgtttctggcttagaacaaaggggcttaattattgatgtt

SEQ ID NO: 10 - protein, 107 aa, Human
MARAALSAAPSNPRLLRVALLLLLLVAAGRRAAGASVATELRCQCLQTLQGIHPKNIQSVNVKSPGPHCAQTEVIATLKNGRKACLNPASPIVKKIIEKMLNSDKSN

SEQ ID NO: 11 - DNA, 794 bp, Human
  misc_feature 7, 14, 22, 35, 37: n = A,T,C or G
atgtgtnataactnagtcaagntcagtgagcattntnagcacattgcctcaacagcttcaaggtgagccagctcaagactttgctctccaccaggcagaagatgacagactgtgaatttggatatatttacaggctggctcaggactatctgcagtgcgtcctacagataccacaacctggatcaggtccaagcaaaacgtccagagtgctacaaaatgttgcgttctcagtccaaaaagaagtggaaaagaatctgaagtcatgcttggacaatgttaatgttgtgtccgtagacactgccagaacactattcaaccaagtgatggaaaaggagtttgaagacgacatcattaactggggaagaattgtaaccatatttgcatttgaaggtattctcatcaagaaacttctacgacagcaaattgccccggatgtggatacctataaggagatttcatattttgttgcggagttcataatgaataacacaggagaatggataaggcaaaacggaggctgggaaaatggctttgtaaagaagtttgaacctaaatctggctggatgacttttctagaagttacaggaaagatctgtgaaatgctatctctcctgaagcaatactgttgaccagaaaggacactccatattgtgaaaccggcctaatttttctgactgatatggaaacgattgccaacacatacttctacttttaaataaacaactttgatgatgtaacttgaccttccagagttatggaaattttgtccccatgtaatggaataaattgtatgt

SEQ ID NO: 12 - protein, 175 aa, Human [sequence omitted]

SEQ ID NO: 13 - DNA, 800 bp, Human
gacgtgaaaatctgccttctcaccatgaggcttctagtcctttccagcctgctctgtatcctgcttctctgcttctccatcttctccacagaagggaagaggcgtcctgccaaggcctggtcaggcaggagaaccaggctctgctgccaccgagtccctagccccaactcaacaaacctgaaaggacatcatgtgaggctctgtaaaccatgcaagcttgagccagagccccgcctttgggtggtgcctggggcactcccacaggtgtagcactcccaaagcaagactccagacagcggagaacctcatgcctggcacctgaggtacccagcagcctcctgtctcccctttcagccttcacagcagtgagctgcaatgttggagggcttcatctcgggctgcaaggaccctgggaaagttccagaactccacgtccttgtctcaattgtgccatcaactttcagagctatcatgagccaacctcaccccacagggcctcagtcgccaccatgtgggcctctccagtgcaaaccaccgagcattccaccatgaccggtcacagctacaaatccagagaccatcaatcctgctagagtgcagggtggcaagcacccaagggtggctgaccaagactgcagagtctcctccatcttcaggtccattcagcctcctggcatttaactaccagcatccagtggtccccaaggaatcccttcctagcctcctgacatgagtctgctggaaagagcatccaaacaaacaagtaataaataaataaataaactcaaaaaaaaaaaaaa

SEQ ID NO: 14 - protein, 81 aa, Human
MRLLVLSSLLCILLLCFSIFSTEGKRRPAKAWSGRRTRLCCHRVPSPNSTNLKGHHVRLCKPCKLEPEPRLWVVPGALPQV

SEQ ID NO: 15 - DNA, 3169 bp, Human
gccaggaataactagagaggaacaatggggttattcagaggttttgttttcctcttagttctgtgcctgctgcaccagtcaaatacttccttcattaagctgaataataatggctttgaagatattgtcattgttatagatcctagtgtgccagaagatgaaaaaataattgaacaaatagaggatatggtgactacagcttctacgtacctgtttgaagccacagaaaaaagattttttttcaaaaatgtatctatattaattcctgagaattggaaggaaaatcctcagtacaaaaggccaaaacatgaaaaccataaacatgctgatgttatagttgcaccacctacactcccaggtagagatgaaccatacaccaagcagttcacagaatgtggagagaaaggcgaatacattcacttcacccctgaccttctacttggaaaaaaacaaaatgaatatggaccaccaggcaaactgtttgtccatgagtgggctcacctccggtggggagtgtttgatgagtacaatgaagatcagcctttctaccgtgctaagtcaaaaaaaatcgaagcaacaaggtgttccgcaggtatctctggtagaaatagagtttataagtgtcaaggaggcagctgtcttagtagagcatgcagaattgattctacaacaaaactgtatggaaaagattgtcaattctttcctgataaagtacaaacagaaaaagcatccataatgtttatgcaaagtattgattctgttgttgaattttgtaacgaaaaaacccataatcaagaagctccaagcctacaaaacataaagtgcaattttagaagtacatgggaggtgattagcaattctgaggattttaaaaacaccatacccatggtgacaccacctcctccacctgtcttctcattgctgaagatcagtcaaagaattgtgtgcttagttcttgataagtctggaagcatggggggtaaggaccgcctaaatcgaatgaatcaagcagcaaaacatttcctgctgcagactgttgaaaatggatcctgggtggggatggttcactttgatagtactgccactattgtaaataagctaatccaaataaaaagcagtgatgaaagaaacacactcatggcaggattacctacatatcctctgggaggaacttccatctgctctggaattaaatatgcatttcaggtgattggagagctacattcccaactcgatggatccgaagtactgctgctgactgatggggaggataacactgcaagttcttgtattgatgaagtgaaacaaagtggggccattgttcattttattgctttgggaagagctgctgatgaagcagtaatagagatgagcaagataacaggaggaagtcatttttatgtttcagatgaagctcagaacaatggcctcattgatgcttttggggctcttacatcaggaaatactgatctctcccagaagtcccttcagctcgaaagtaagggattaacactgaatagtaatgcctggatgaacgacactgtcataattgatagtacagtgggaaaggacacgttctttctcatcacatggaacagtctgcctcccagtatttctctctgggatcccagtggaacaataatggaaaatttcacagtggatgcaacttccaaaatggcctatctcagtattccaggaactgcaaaggtgggcacttgggcatacaatcttcaagccaaagcgaacccagaaacattaactattacagtaacttctcgagcagcaaattcttctgtgcctccaatcacagtgaatgctaaaatgaataaggacgtaaacagtttccccagcccaatgattgtttacgcagaaattctacaaggatatgtacctgttcttggagccaatgtgactgctttcattgaatcacagaatggacatacagaagttttggaacttttggataatggtgcaggcgctgattctttcaagaatgatggagtctactccaggtattttacagcatatacagaaaatggcagatatagcttaaaagttcgggctcatggaggagcaaacactgccaggctaaaattacggcctccactgaatagagccgcgtacataccaggctgggtagtgaacggggaaattgaagcaaacccgccaagacctgaaattgatgaggatactcagaccaccttggaggatttcagccgaacagcatccggaggtgcatttgtggtatcacaagtcccaagccttcccttgcctgaccaatacccaccaagtcaaatcacagaccttgatgccacagttcatgaggataagattattcttacatggacagcaccaggagataattttgatgttggaaaagttcaacgttatatcataagaataagtgcaagtattcttgatctaagagacagttttgatgatgctcttcaagtaaatactactgatctgtcaccaaaggaggccaactccaaggaaagctttgcatttaaaccagaaaatatctcagaagaaaatgcaacccacatatttattgccattaaaagtatagataaaagcaatttgacatcaaaagtatccaacattgcacaagtaactttgtttatccctcaagcaaatcctgatgacattgatcctactcctactcctactcctactcctgataaaagtcataattctggagttaatatttctacgctggtattgtctgtgattgggtctgttgtaattgttaactttattttaagtaccaccatttgaaccttaacgaagaaaaaaatcttcaagtagacctagaagagagttttaaaaaacaaaacaatgtaagtaaaggatatttctgaatcttaaaattcatcccatgtgtgatcataaactcataaaaataattttaagatgtcggaaaaggatactttgattaaataaaaacactcatggatatgtaaaaactgtcaagattaaaatttaatagtttcatttatttgttattttatttgtaagaaatagtgatgaacaaagatcctttttcatactgatacctggttgtatattatttgatgcaacagttttctgaaatgatatttcaaattgcatcaagaaattaaaatcatctatctgagtagtcaaaatacaagtaaaggagagcaaataa

SEQ ID NO: 16 - protein, 917 aa, Human
MGLFRGFVFLLVLCLLHQSNTSFIKLNNNGFEDIVIVIDPSVPEDEKIIEQIEDMVTTASTYLFEATEKRFFFKNVSILIPENWKENPQYKRPKHENHKHADVIVAPPTLPGRDEPYTKQFTECGEKGEYIHFTPDLLLGKKQNEYGPPGKLFVHEWAHLRWGVFDEYNEDQPFYRAKSKKIEATRCSAGISGRNRVYKCQGGSCLSRACRIDSTTKLYGKDCQFFPDKVQTEKASIMFMQSIDSVVEFCNEKTHNQEAPSLQNIKCNFRSTWEVISNSEDFKNTIPMVTPPPPPVFSLLKISQRIVCLVLDKSGSMGGKDRLNRMNQAAKHFLLQTVENGSWVGMVHFDSTATIVNKLIQIKSSDERNTLMAGLPTYPLGGTSICSGIKYAFQVIGELHSQLDGSEVLLLTDGEDNTASSCIDEVKQSGAIVHFIALGRAADEAVIEMSKITGGSHFYVSDEAQNNGLIDAFGALTSGNTDLSQKSLQLESKGLTLNSNAWMNDTVIIDSTVGKDTFFLITWNSLPPSISLWDPSGTIMENFTVDATSKMAYLSIPGTAKVGTWAYNLQAKANPETLTITVTSRAANSSVPPITVNAKMNKDVNSFPSPMIVYAEILQGYVPVLGANVTAFIESQNGHTEVLELLDNGAGADSFKNDGVYSRYFTAYTENGRYSLKVRAHGGANTARLKLRPPLNRAAYIPGWVVNGEIEANPPRPEIDEDTQTTLEDFSRTASGGAFVVSQVPSLPLPDQYPPSQITDLDATVHEDKIILTWTAPGDNFDVGKVQRYIIRISASILDLRDSFDDALQVNTTDLSPKEANSKESFAFKPENISEENATHIFIAIKSIDKSNLTSKVSNIAQVTLFIPQANPDDIDPTPTPTPTPDKSHNSGVNISTLVLSVIGSVVIVNFILSTTI

SEQ ID NO: 17 - DNA, 737 bp, Human
ctcagccttcaggccactcagctggtgccaaatagagtagggatgagctgtccccacagagacctgcccagtgcacattgtgagaactggaagtttccagggggctgctttgcatctgaaactgtcagccccagaatgttgacagtcgctctcctagcccttctctgtgcctcagcctctggcaatgccattcaggccaggtcttcctcctatagtggagagtatggaagtggtggtggaaagcgattctctcattctggcaaccagttggacggccccatcaccgccctccgggtccgagtcaacacatactacatcgtaggtcttcaggtgcgctatggcaaggtgtggagcgactatgtgggtggtcgcaacggagacctggaggagatctttctgcaccctggggaatcagtgatccaggtttctgggaagtacaagtggtacctgaagaagctggtatttgtgacagacaagggccgctatctgtcttttgggaaagacagtggcacaagtttcaatgccgtccccttgcaccccaacaccgtgctccgcttcatcagtggccggtctggttctctcatcgatgccattggcctgcactgggatgtttaccccactagctgcagcagatgctgagcctcctctccttggcaggggcactgtgatgaggagtaagaactcccttatcactaacccccatccaaatggctcaataaaaaaatatggttaaggct

SEQ ID NO: 18 - protein, 198 aa, Human
MSCPHRDLPSAHCENWKFPGGCFASETVSPRMLTVALLALLCASASGNAIQARSSSYSGEYGSGGGKRFSHSGNQLDGPITALRVRVNTYYIVGLQVRYGKVWSDYVGGRNGDLEEIFLHPGESVIQVSGKYKWYLKKLVFVTDKGRYLSFGKDSGTSFNAVPLHPNTVLRFISGRSGSLIDAIGLHWDVYPTSCSRC

SEQ ID NO: 19 - DNA, 2879 bp, Human
tgagtggatggacactgcctcttagaactagaacttagaactttatcttgaaaatgtaccactgttgcagaagctcctcacagagtatgtgtcaggcatttttaacctgctaaaggcaagaagaagtgttcaccacatagttgcaaaggtcttcaacttgccacagccaacagaaaaatcaaaatgattgaaccctttgggaatcagtatattgtggccaggccagtgtattctacaaatgcttttgaggaaaatcataaaaagacaggaagacatcataagacatttctggatcatctcaaagtgtgttgtagctgttccccacaaaaggccaagagaattgtcctctctttgttccccatagcatcttggttgccagcataccggcttaaagaatggttgctcagtgatattgtttctggtatcagcacagggattgtggccgtactacaaggtttagcatttgctctgctggtcgacattcccccagtctatgggttgtatgcatcctttttcccagccataatctaccttttcttcggcacttccagacacatatccgtgggtccgtttccgattctgagtatgatggtgggactagcagtttcaggagcagtttcaaaagcagtcccagatcgcaatgcaactactttgggattgcctaacaactcgaataattcttcactactggatgacgagagggtgagggtggcggcggcggcatcagtcacagtgctttctggaatcatccagttggcttttgggattctgcggattggatttgtagtgatatacctgtctgagtccctcatcagtggcttcactactgctgctgctgttcatgttttggtttcccaactcaaattcatttttcagttgacagtcccgtcacacactgatccagtttcaattttcaaagtactatactctgtattctcacaaatagagaagactaatattgcagacctggtgacagctctgattgtccttttggttgtatccattgttaaagaaataaatcagcgcttcaaagacaaacttccagtgcccattccaatcgaattcattatgaccgtgattgcagcaggtgtatcctacggctgtgactttaaaaacaggtttaaagtggctgtggttggggacatgaatcctggatttcagccccctattacacctgacgtggagactttccaaaacaccgtaggagattgcttcggcatcgcaatggttgcatttgcagtggccttttcagttgccagcgtctattccctcaaatacgattatccacttgatggcaatcaggagttaatagccttgggactgggtaacatagtctgtggagtattcagaggatttgctgggagtactgccctctccagatcagcagttcaggagagcacaggaggcaaaacacagattgctgggcttattggtgccatcatcgtgctgattgtcgttctagccattggatttctcctggcgcctctacaaaagtccgtcctggcagctttagcattgggaaacttaaagggaatgctgatgcagtttgctgaaataggcagattgtggcgaaaggacaaatatgattgtttaatttggatcatgaccttcatcttcaccattgtcctgggactcgggttaggcctggcagctagtgtggcatttcaactgctaaccatcgtgttcaggacccaatttccaaaatgcagcacgctggctaatattggaagaaccaacatctataagaataaaaaagattattatgatatgtatgagccagaaggagtgaaaattttcagatgtccatctcctatctactttgcaaacattggtttctttaggcggaaacttatcgatgctgttggctttagtccacttcgaattctacgcaagcgcaacaaagctttgaggaaaatccgaaaactgcagaagcaaggcttgctacaagtgacaccaaaaggatttatatgtactgttgacaccataaaagattctgacgaagagctggacaacaatcagatagaagtactggaccagccaatcaataccacagacctgcctttccacattgactggaatgatgatcttcctctcaacattgaggtccccaaaatcagcctccacagcctcattctcgacttttcagcagtgtcctttcttgatgtttcttcagtgaggggccttaaatcgattttgcaagaatttatcaggatcaaggtagatgtgtatatcgttggaactgatgatgacttcattgagaagcttaaccggtatgaattttttgatggtgaagtgaaaagctcaatatttttcttaacaatccatgatgctgttttgcatattttgatgaagaaagattacagtacttcaaagtttaatcccagtcaggaaaaagatggaaaaattgattttaccataaatacaaatggaggattacgtaatcgggtatatgaggtgccagttgaaacaaaattctaatcaacatataattcagaaggatcttcatctgactatgacataaaaacaactttatacccagaaagttattgataagttcatacattgtacgaagagtatttttgacagaatatgtttcaaactttggaacaagatggttctagcatggcatatttttcacatatctagtatgaaattatataagtattctaaattttatatcttgtagctttatcaaagggtgaaaattattttgttcatacatatttttgtagcactgacagatttccatcctagtcactaccttcatgcataggtttagcagtatagtggcgccactgttttgaatctcataatttatacaggtcatattaatatatttccattaaaaaatcagttgtacagtgaaaaaaaaaaaaaaa

SEQ ID NO: 20 - protein, 764 aa, Human
MIEPFGNQYIVARPVYSTNAFEENHKKTGRHHKTFLDHLKVCCSCSPQKAKRIVLSLFPIASWLPAYRLKEWLLSDIVSGISTGIVAVLQGLAFALLVDIPPVYGLYASFFPAIIYLFFGTSRHISVGPFPILSMMVGLAVSGAVSKAVPDRNATTLGLPNNSNNSSLLDDERVRVAAAASVTVLSGIIQLAFGILRIGFVVIYLSESLISGFTTAAAVHVLVSQLKFIFQLTVPSHTDPVSIFKVLYSVFSQIEKTNIADLVTALIVLLVVSIVKEINQRFKDKLPVPIPIEFIMTVIAAGVSYGCDFKNRFKVAVVGDMNPGFQPPITPDVETFQNTVGDCFGIAMVAFAVAFSVASVYSLKYDYPLDGNQELIALGLGNIVCGVFRGFAGSTALSRSAVQESTGGKTQIAGLIGAIIVLIVVLAIGFLLAPLQKSVLAALALGNLKGMLMQFAEIGRLWRKDKYDCLIWIMTFIFTIVLGLGLGLAASVAFQLLTIVFRTQFPKCSTLANIGRTNIYKNKKDYYDMYEPEGVKIFRCPSPIYFANIGFFRRKLIDAVGFSPLRILRKRNKALRKIRKLQKQGLLQVTPKGFICTVDTIKDSDEELDNNQIEVLDQPINTTDLPFHIDWNDDLPLNIEVPKISLHSLILDFSAVSFLDVSSVRGLKSILQEFIRIKVDVYIVGTDDDFIEKLNRYEFFDGEVKSSIFFLTIHDAVLHILMKKDYSTSKFNPSQEKDGKIDFTINTNGGLRNRVYEVPVETKF

SEQ ID NO: 21 - DNA, 655 bp, Human
cagtaacctgccctctttaaaagtcccgccgcttccccctggcatccacaacagccacccctctctcgggcactgctgccatgaatgccttcctgctcttcgcactgtgcctccttggggcctgggccgccttggcaggaggggtcaccgtgcaggatggaaatttctccttttctctggagtcagtgaagaagctcaaagacctccaggagccccaggagcccagggttgggaaactcaggaactttgcacccatccctggtgaacctgtggttcccatcctctgtagcaacccgaactttccagaagaactcaagcctctctgcaaggagcccaatgcccaggagatacttcagaggctggaggaaatcgctgaggacccgggcacatgtgaaatctgtgcctacgctgcctgtaccggatgctaggggggcttgcccactgcctgcctcccctccgcagcagggaagctcttttctcctgcagaaagggccacccatgatactccactcccagcagctcaacctaccctggtccagtcgggaggagcagcccggggaggaactgggtgactggaggcctcgccccaacactgtccttccctgccacttcaacccccagctaataaaccagattccagagtaaaaaaaaaaaa

SEQ ID NO: 22 - protein, 115 aa, Human
MNAFLLFALCLLGAWAALAGGVTVQDGNFSFSLESVKKLKDLQEPQEPRVGKLRNFAPIPGEPVVPILCSNPNFPEELKPLCKEPNAQEILQRLEEIAEDPGTCEICAYAACTGC

SEQ ID NO: 23 - DNA, 1244 bp, Human
cagtcctcaggtgcaacccctgcgtggtctctgtggcagccttctctcattcagagcttgcacagttgcagttagttattccaggtattatttttgttttcagaaaaagaaaactcagtagaagataatggcaagtccagactggggatatgatgacaaaaatggtcctgaacaatggagcaagctgtatcccattgccaatggaaataaccagtcccctgttgatattaaaaccagtgaaaccaaacatgacacctctctgaaacctattagtgtctcctacaacccagccacagccaaagaaattatcaatgtggggcattccttccatgtaaattttgaggacaacgataaccgatcagtgctgaaaggtggtcctttctctgacagctacaggctctttcagttccattttcactggggcagtacaaatgagcatggttcagaacatacagtggatggagtcaaatattctgccgagcttcacgtagctcactggaattctgcaaagtactccagccttgctgaagctgcctcaaaggctgatggtttggcagttattggtgttttgatgaaggttggtgaggccaacccaaagctgcagaaagtacttgatgccctccaagcaattaaaaccaagggcaaacgagccccattcacaaattttgacccctctactctccttccttcatccctggatttctggacctaccctggctctctgactcatcctcctctttatgagagtgtaacttggatcatctgtaaggagagcatcagtgtcagctcagagcagctggcacaattccgcagccttctatcaaatgttgaaggtgataacgctgtccccatgcagcacaacaaccgcccaacccaacctctgaagggcagaacagtgagagcttcattttgatgattctgagaagaaacttgtccttcctcaagaacacagccctgcttctgacataatccagtaaaataataatttttaagaaataaatttatttcaatattagcaagacagcatgccttcaaatcaatctgtaaaactaagaaacttaaattttagttcttactgcttaattcaaataataattagtaagctagcaaatagtaatctgtaagcataagcttatgcttaaattcaagtttagtttgaggaattctttaaaattacaactaagtgatttgtatgtctatttttttcagtttatttgaaccaataaaataattttatctctttc

SEQ ID NO: 24 - protein, 261 aa, Human
MASPDWGYDDKNGPEQWSKLYPIANGNNQSPVDIKTSETKHDTSLKPISVSYNPATAKEIINVGHSFHVNFEDNDNRSVLKGGPFSDSYRLFQFHFHWGSTNEHGSEHTVDGVKYSAELHVAHWNSAKYSSLAEAASKADGLAVIGVLMKVGEANPKLQKVLDALQAIKTKGKRAPFTNFDPSTLLPSSLDFWTYPGSLTHPPLYESVTWIICKESISVSSEQLAQFRSLLSNVEGDNAVPMQHNNRPTQPLKGRTVRASF

SEQ ID NO: 25 - DNA, 3111 bp, Human
cggctcgaggaaatcacagggagatgtacagcaatggggccatttaagagttctgtgttcatcttgattcttcaccttctagaaggggccctgagtaattcactcattcagctgaacaacaatggctatgaaggcattgtcgttgcaatcgaccccaatgtgccagaagatgaaacactcattcaacaaataaaggacatggtgacccaggcatctctgtatctgtttgaagctacaggaaagcgattttatttcaaaaatgttgccattttgattcctgaaacatggaagacaaaggctgactatgtgagaccaaaacttgagacctacaaaaatgctgatgttctggttgctgagtctactcctccaggtaatgatgaaccctacactgagcagatgggcaactgtggagagaagggtgaaaggatccacctcactcctgatttcattgcaggaaaaaagttagctgaatatggaccacaaggtagggcatttgtccatgagtgggctcatctacgatggggagtatttgacgagtacaataatgatgagaaattctacttatccaatggaagaatacaagcagtaagatgttcagcaggtattactggtacaaatgtagtaaagaagtgtcagggaggcagctgttacaccaaaagatgcacattcaataaagtaacaggactctatgaaaaaggatgtgagtttgttctccaatcccgccagacggagaaggcttctataatgtttgcacaacatgttgattctatagttgaattctgtacagaacaaaaccacaacaaagaagctccaaacaagcaaaatcaaaaatgcaatctccgaagcacatgggaagtgatccgtgattctgaggactttaagaaaaccactcctatgacaacacagccaccaaatcccaccttctcattgctgcagattggacaaagaattgtgtgtttagtccttgacaaatctggaagcatggcgactggtaaccgcctcaatcgactgaatcaagcaggccagcttttcctgctgcagacagttgagctggggtcctgggttgggatggtgacatttgacagtgctgcccatgtacaaagtgaactcatacagataaacagtggcagtgacagggacacactcgccaaaagattacctgcagcagcttcaggagggacgtccatctgcagcgggcttcgatcggcatttactgtgattaggaagaaatatccaactgatggatctgaaattgtgctgctgacggatggggaagacaacactataagtgggtgctttaacgaggtcaaacaaagtggtgccatcatccacacagtcgctttggggccctctgcagctcaagaactagaggagctgtccaaaatgacaggaggtttacagacatatgcttcagatcaagttcagaacaatggcctcattgatgcttttggggccctttcatcaggaaatggagctgtctctcagcgctccatccagcttgagagtaagggattaaccctccagaacagccagtggatgaatggcacagtgatcgtggacagcaccgtgggaaaggacactttgtttcttatcacctggacaacgcagcctccccaaatccttctctgggatcccagtggacagaagcaaggtggctttgtagtggacaaaaacaccaaaatggcctacctccaaatcccaggcattgctaaggttggcacttggaaatacagtctgcaagcaagctcacaaaccttgaccctgactgtcacgtcccgtgcgtccaatgctaccctgcctccaattacagtgacttccaaaacgaacaaggacaccagcaaattccccagccctctggtagtttatgcaaatattcgccaaggagcctccccaattctcagggccagtgtcacagccctgattgaatcagtgaatggaaaaacagttaccttggaactactggataatggagcaggtgctgatgctactaaggatgacggtgtctactcaaggtatttcacaacttatgacacgaatggtagatacagtgtaaaagtgcgggctctgggaggagttaacgcagccagacggagagtgataccccagcagagtggagcactgtacatacctggctggattgagaatgatgaaatacaatggaatccaccaagacctgaaattaataaggatgatgttcaacacaagcaagtgtgtttcagcagaacatcctcgggaggctcatttgtggcttctgatgtcccaaatgctcccatacctgatctcttcccacctggccaaatcaccgacctgaaggcggaaattcacgggggcagtctcattaatctgacttggacagctcctggggatgattatgaccatggaacagctcacaagtatatcattcgaataagtacaagtattcttgatctcagagacaagttcaatgaatctcttcaagtgaatactactgctctcatcccaaaggaagccaactctgaggaagtctttttgtttaaaccagaaaacattacttttgaaaatggcacagatcttttcattgctattcaggctgttgataaggtcgatctgaaatcagaaatatccaacattgcacgagtatctttgtttattcctccacagactccgccagagacacctagtcctgatgaaacgtctgctccttgtcctaatattcatatcaacagcaccattcctggcattcacattttaaaaattatgtggaagtggataggagaactgcagctgtcaatagcctagggctgaatttttgtcagataaataaaataaatcattcatccttttttttgattataaaattttctaaaatgtattttagacttcctgtagggggcgatatactaaatgtatatagtacatttatactaaatgtattcctgtagggggcgatatactaaatgtattttagacttcctgtagggggcgataaaataaaatgctaaacaactgggtatacatgcataaaaactatccattcaaacccaaaaatttaataatcattgagtcttttattaatgaatttgaatactagaaagaaacagggcttgcatcaataaatggaagtatgagtgt

SEQ ID NO: 26 - protein, 914 aa, Human [sequence omitted]

SEQ ID NO: 27 - DNA, 1756 bp, Human
caaatgagtgctgttaaagttcctccaggaaacttcagcagagaaaaacatttgcttcacatctcatcaaatcttctgcatcaagccacatcatgttaaacaaccttctgctgttctcccttcagataagtctcataggaaccactcttggtgggaatgttttgatttggccaatggaaggtagtcattggctaaatgttaagataattatagatgagctcattaaaaaggagcataatgtgactgtcctagttgcctctggtgcacttttcatcacaccaacctctaacccatctctgacatttgaaatatataaggtgccctttggcaaagaaagaatagaaggagtaattaaggacttcgttttgacatggctggaaaatagaccatctccttcaaccatttggagattctatcaggagatggccaaagtaatcaaggacttccacatggtgtctcaggagatctgtgatggcgttcttaaaaaccaacagctgatggcaaagctaaagaaaagcaagtttgaagtcctggtgtctgatccagtatttccttgtggcgatatagtagctttaaaacttggaattccatttatgtactccttgaggttttctccagcctcaacagtggaaaagcactgtgggaaggtaccataccctccttcctatgttcctgctgttttatcagaactcaccgaccaaatgtctttcactgacagaataagaaatttcatctcctaccacctacaggactacatgtttgaaactctttggaaatcatgggattcatactatagtaaagctttaggaagacccactacgttatgtgagactatggggaaagctgaaatttggttaatccgaacatattgggattttgaatttcctcgtccatacttacctaattttgagtttgttggaggattgcactgcaaacctgccaaacctttacctaaggaaatggaagaatttatccagagctcaggtaaaaatggtgttgtggtgttttctctgggatcaatggtcaaaaaccttacagaagaaaaggccaatcttattgcctcagcccttgcccagattccacagaaggttttatggagatacaaaggaaagaaaccagccacattaggaaacaatactcagctctttgattggataccccagaatgatcttcttggacatcccaaaaccaaagcttttatcactcatggtggaactaatgggatctacgaagctatttaccacggagtccctatggtgggagttcccatgtttgctgatcagcctgataacattgctcacatgaaggccaaaggagcagctgtggaagtgaacctaaacacaatgacaagtgtggatttgcttagcgctttgagaacagtcattaatgaaccttcttataaagagaatgctatgaggttatcaagaattcaccatgatcaacctgtaaagcccctggatcgagcagtcttctggatcgagtttgtcatgcgccacaaaggagccaagcaccttcgggttgcagcccatgacctcacctggttccagtaccactctttggatgtaattgggttcttgctggtctgtgtgacaacggctatatttttggtcatacaatgttgtttgttttcctgtcaaaaatttggtaagataggaaagaagaaaaaaagagaataggtcaagaaaaagaggaaatatatatatttttaagtttggcaaaatcctgagtagtggaagtcctattaattccagacaaa

SEQ ID NO: 28 - protein, 527 aa, Human
MLNNLLLFSLQISLIGTTLGGNVLIWPMEGSHWLNVKIIIDELIKKEHNVTVLVASGALFITPTSNPSLTFEIYKVPFGKERIEGVIKDFVLTWLENRPSPSTIWRFYQEMAKVIKDFHMVSQEICDGVLKNQQLMAKLKKSKFEVLVSDPVFPCGDIVALKLGIPFMYSLRFSPASTVEKHCGKVPYPPSYVPAVLSELTDQMSFTDRIRNFISYHLQDYMFETLWKSWDSYYSKALGRPTTLCETMGKAEIWLIRTYWDFEFPRPYLPNFEFVGGLHCKPAKPLPKEMEEFIQSSGKNGVVVFSLGSMVKNLTEEKANLIASALAQIPQKVLWRYKGKKPATLGNNTQLFDWIPQNDLLGHPKTKAFITHGGTNGIYEAIYHGVPMVGVPMFADQPDNIAHMKAKGAAVEVNLNTMTSVDLLSALRTVINEPSYKENAMRLSRIHHDQPVKPLDRAVFWIEFVMRHKGAKHLRVAAHDLTWFQYHSLDVIGFLLVCVTTAIFLVIQCCLFSCQKFGKIGKKKKRE

SEQ ID NO: 29 - DNA, 1870 bp, Human
actcccctccgaggggtctgaccacgcttgggccgagtcatacgcccacgcgtccgggacctcctgccctcaggtgatccatccacctcggccagtcaaagtgctgggattacaggcatgagccattgcacccagccgatactactatatccccattttacagatgagcacatgggcaaattgagggtaaggcactgacccatgatcatacagctgagaagtggcaaaggcaggatttgaacctagaacctctggctccacacactagtaatctaaaccactctccctacaatacaacatacgtggtaaagatgtgtggtgggcacgcaatcaacgtaggtcccttcacagttgctgggagaggcaggaatttgcagttcctccgcgttctcctcctccgctgcccacctgtcctgggtcattcctgcagcctgccctgccctgcctggtctcaccctccctctgccaacagaagtctgggcagggttttatgggctctgataaggccctggcagggccgaagttcatgagcacttcctctttgcaggagggcgtaggggaggggacccaggtgatttgggtcctggctggtcaccagggaagctggcaagggaagggagactagggtgcgctctaggagaagccgacagcctgagagtcccagaagaggagccctgtggaccctcccctgccagccactcccttaccctgggtataagagccaccaccgcctgccatccgccaccatctcccactcctgcagctcttctcacaggaccagccactagcgcagcctcgagcgatggcctatgtccccgcaccgggctaccagcccacctacaacccgacgctgccttactaccagcccatcccgggcgggctcaacgtgggaatgtctgtttacatccaaggagtggccagcgagcacatgaagcggttcttcgtgaactttgtggttgggcaggatccgggctcagacgtcgccttccacttcaatccgcggtttgacggctgggacaaggtggtcttcaacacgttgcagggcgggaagtggggcagcgaggagaggaagaggagcatgcccttcaaaaagggtgccgcctttgagctggtcttcatagtcctggctgagcactacaaggtggtggtaaatggaaatcccttctatgagtacgggcaccggcttcccctacagatggtcacccacctgcaagtggatggggatctgcaacttcaatcaatcaacttcatcggaggccagcccctccggccccagggacccccgatgatgccaccttaccctggtcccggacattgccatcaacagctgaacagcctgcccaccatggaaggacccccaaccttcaacccgcctgtgccatatttcgggaggctgcaaggagggctcacagctcgaagaaccatcatcatcaagggctatgtgcctcccacaggcaagagctttgctatcaacttcaaggtgggctcctcaggggacatagctctgcacattaatccccgcatgggcaacggtaccgtggtccggaacagccttctgaatggctcgtggggatccgaggagaagaagatcacccacaacccatttggtcccggacagttctttgatctgtccattcgctgtggcttggatcgcttcaaggtttacgccaatggccagcacctctttgactttgcccatcgcctctcggccttccagagggtggacacattggaaatccagggtgatgtcaccttgtcctatgtccagatctaatctattcctggggccataactcatgggaaaacagaattatcccctaggactcctttctaagcccctaataaaatgtctgagggtgaaaaaaaaaaa

SEQ ID NO: 30 - protein, 323 aa, Human
MAYVPAPGYQPTYNPTLPYYQPIPGGLNVGMSVYIQGVASEHMKRFFVNFVVGQDPGSDVAFHFNPRFDGWDKVVFNTLQGGKWGSEERKRSMPFKKGAAFELVFIVLAEHYKVVVNGNPFYEYGHRLPLQMVTHLQVDGDLQLQSINFIGGQPLRPQGPPMMPPYPGPGHCHQQLNSLPTMEGPPTFNPPVPYFGRLQGGLTARRTIIIKGYVPPTGKSFAINFKVGSSGDIALHINPRMGNGTVVRNSLLNGSWGSEEKKITHNPFGPGQFFDLSIRCGLDRFKVYANGQHLFDFAHRLSAFQRVDTLEIQGDVTLSYVQI

(19) United States
(12) Patent Application Publication
(10) Pub. No.: US 2003/0101002 A1
(43) Pub. Date: May 29, 2003

(54) Methods for analyzing gene expression patterns

(76) Inventors: Gabor T. Bartha, Mountain View, CA (US); Michael Walker, Sunnyvale, CA (US)

Correspondence Address: BEYER WEAVER & THOMAS LLP, P.O. BOX 778, BERKELEY, CA, 94704-0778, US

(21) Appl. No.: 10/235,994

(22) Filed: Sep. 4, 2002

Related U.S. Application Data
(63) Continuation-in-part of application No. 10/003,608, filed on Nov. 1, 2001.
(60) Provisional application No. 60/245,081, filed on Nov. 1, 2000.

(51) Int. Cl. C12Q 1/68; G06F 19/00; G01N 33/48; G01N 33/50; G06K 9/00

(52) U.S. Cl. 702/20; 435/6; 382/128

(57) ABSTRACT

The invention provides novel disease-associated genes and polypeptides encoded by those genes. The invention also provides expression vectors, host cells, antibodies, agonists, and antagonists. The invention also provides methods for diagnosing, treating or preventing diseases.

### RELATED APPLICATIONS

This application is a continuation-in-part of U.S. Ser. No. 10/003,608, filed Nov. 1, 2001, from which priority under 35 U.S.C. §120 is claimed, which is incorporated by reference in its entirety for all purposes. This application also claims priority under 35 U.S.C. §119(e) to U.S. Ser. No. 60/245,081, filed Nov. 1, 2000, which is incorporated by reference in its entirety for all purposes.

### BACKGROUND OF THE INVENTION

The present invention generally relates to systems and methods for facilitating the identification of disease associated genes. In particular, the invention relates to improved techniques for analyzing gene expression patterns to discover disease associated genes. The invention also relates to three novel cancer-associated genes identified by the method and their corresponding polypeptides and to the use of these biomolecules in diagnosis, prognosis, treatment, prevention, and evaluation of therapies for diseases, particularly diseases associated with cell proliferation, such as cancer.

The DNA sequences of many human genes have been determined, but for many of these genes, their biological function, and in particular their relationship to disease, is unknown or poorly understood. Current laboratory and computational methods to determine new methods that provide additional information on function are desirable.

The recent development of complementary DNA micro-array technology provides a powerful analytical tool for human genetic research (M. Schena, D. Shalon, R. W. Davis, and P. O. Brown, “Quantitative monitoring of gene expression patterns with a complementary DNA microarray,” Science, 270(5235), 467-70, 1995). One of its basic applications is to quantitatively analyze fluorescence signals that represent the relative abundance of mRNA from two distinct tissue samples. cDNA micro-arrays are prepared by automatically printing thousands of cDNAs in an array format on glass microscope slides, which provide gene-specific hybridization targets. Two different samples (of mRNA) can be labeled with different fluors and then co-hybridized on to each arrayed gene. Ratios of gene-expression levels between the samples are calculated and used to detect meaningfully different expression levels between the samples for a given gene. Such monitoring technologies have been applied to the identification of genes which are up regulated or down regulated in various diseased or physiological states, the analyses of members of signaling cellular states, and the identification of targets for various drugs.

The various characteristics of this analytic scheme make it particularly useful for directly comparing the abundance of mRNAs present in two cell types. Visual inspection of such a comparison is sufficient to find genes where there is a very large differential rate of expression.

Walker et al. (1999) Genome Research 91:1198-1203 discusses a method for identifying genes associated with disease wherein the expression of genes in multiple cDNA libraries was examined. The method described therein allows one to perform a coexpression analysis on clone count data from sequencing. The statistical analysis is performed using a categorical method (i.e., present or absent in clone count data from a library) rather than analyzing expression as a continuous variable using linear or rank correlation.

For single channel microarray data, one could conceivably define a threshold of detection and use the same categories as described in Walker. However, typically Pearson's or Spearman's correlational methods are used for the analysis of single channel microarray data because of risk of effective information loss resulting from converting real valued data to categories.

As with single channel, it is also not practical to categorize data for dual channel microarray data as present or absent. In addition, each channel of dual channel technology is not absolute; thus, further increasing the difficulty in defining the threshold. Moreover, the categories of absent or present are not appropriate when applied to channel ratios.

A more thorough study of the changes in expression requires the ability to discern more subtle changes in expression level and the ability to determine whether observed differences are the result of random variation or whether they are likely to be meaningful changes. As such, there continues to be interest in the development of new methodologies of gene expression analysis, particularly for methodologies applicable to either single channel or dual channel microarray technology.

### SUMMARY OF THE INVENTION

In one aspect, the present invention provides a method for identifying biomolecules, such as polynucleotides or polypeptides, useful in the diagnosis, prognosis, treatment, prevention, and evaluation of therapies for diseases. The method can also be employed for elucidating genes involved in a common regulatory pathway.

The method comprises first characterizing expression patterns of polynucleotides and more particularly, mRNAs. The expressed polynucleotides comprise genes of known and unknown functions. The expression patterns can be obtained through the analysis of a plurality of dual channel microarray data or through single channel data using a defined threshold. Second, the expression patterns of one or more function-specific genes are compared with the expression patterns of one or more of the genes of unknown function to identify a subset of novel genes which have similar expression patterns to those of the function-specific genes.

The method compares the expression pattern of two genes by first generating an expression data vector for each gene. The vector comprises entries for each gene wherein a differentially expressed gene is represented by a one and a non-differentially expressed gene by a zero. The vectors are then analyzed to determine whether the expression patterns of any of the genes are similar. Expression patterns are similar if a particular probability threshold is met. Preferably, the probability threshold is less than 10⁻⁷, and more preferably less than 10^{-9.}

In a preferred embodiment, the function-specific genes are disease-specific gene sequences including TNF-inducible chemokines, including human tumor necrosis factor alpha inducible protein A20; human cytokine (GRO-beta) mRNA; human IL-8; human GRO (growth regulated) gene; and human mRNA for GRS protein. Other disease-specific gene sequences include those involved with cancer of the digestive tract and/or colon, such as those listed in Table 4. These groups of disease-specific genes are used to identify other polynucleotides of unidentified function that are predominantly coexpressed with the disease-specific genes. The polynucleotides analyzed by the present invention can be expressed sequence tags (ESTs), assembled sequences, full length gene coding sequences, introns, regulatory regions, 5′ untranslated regions, 3′ untranslated regions and the like.

In a second aspect, the invention entails a substantially purified polynucleotide identified by the method of the present invention as being associated with cancer. In particular, the polynucleotide comprises a sequence selected from the group consisting of SEQ ID NOs:7, 13, or 17 or its complement or a variant having at least 70% sequence identity to SEQ ID NOs: 7, 13, or 17 or a polynucleotide that hybridizes under stringent conditions to SEQ ID NOs: 7, 13, or 17 or a polynucleotide encoding SEQ ID NOs: 8, 14, or 18. The present invention also entails a polynucleotide comprising at least 18 consecutive nucleotides of a sequence provided above. The polynucleotide is suitable for use in diagnosis, treatment, prognosis, or prevention of a cancer. The polynucleotide is also suitable for the evaluation of therapies for cancer.

In another aspect, the invention provides an expression vector comprising a polynucleotide described above, a host cell comprising the expression vector, and a method for detecting a target polynucleotide in a sample.

In a further aspect, the invention provides a substantially purified polypeptide comprising an amino acid sequence selected from the group consisting of SEQ ID NO:8, SEQ ID NO:14, and SEQ ID NO:16. The invention also provides a substantially purified polypeptide having at least 85% identity to SEQ ID NOs:8, 14, or 18. Additionally, the invention also provides a sequence with at least 6 sequential amino acids of SEQ ID NOs:8, 14, or 18.

The invention also provides a method for producing a substantially purified polypeptide comprising the amino acid sequence referred to above, and antibodies, agonists, and antagonists which specifically bind to the polypeptide. Pharmaceutical compositions comprising the polynucleotides or polypeptides of the invention are also contemplated. Methods for producing a polypeptide of the invention and methods for detecting a target polynucleotide complementary to a polynucleotide of the invention are also included.

### BRIEF DESCRIPTION OF THE DRAWINGS

The accompanying drawings, which are incorporated in and form a part of this specification, illustrate embodiments of the invention and, together with the description, serve to explain the principles of the invention.

FIG. 1 shows a high level process flow for identifying novel genes that exhibit a statistically significant co-differential expression pattern with a target gene.

FIG. 2 is a block diagram of a computer system that may be used to implement various aspects of this invention such as the algorithms for comparing expression patterns.

FIG. 3 depicts—at a high level—processes of the invention utilizing either single channel or dual channel data.

### BRIEF DESCRIPTION OF THE SEQUENCE LISTING

The Sequence Listing, which is incorporated herein by reference in its entirety, provides exemplary disease-associated sequences including polynucleotide sequences, SEQ ID NOs: 7, 13, or 17, and polypeptide sequences, SEQ ID NOs: 8, 14, or 18. Each sequence is identified by a sequence identification number (SEQ ID NO) and/or by the Incyte Clone number from which the sequence was first identified.

### DETAILED DESCRIPTION OF EXAMPLE EMBODIMENTS

Reference will now be made in detail to the preferred embodiments of the invention. While the invention will be described in conjunction with preferred embodiments, it should be understood that such embodiments are not intended to limit the invention to these embodiments. On the contrary, the invention is intended to cover alternatives, modifications and equivalents which are included within the spirit and scope of the invention. For example, the invention will be described by referring to embodiments providing methods, compositions, data analysis systems and computer program products for discovering functional regions in a genome. However, the methods, compositions, computational analysis and computer program products may be useful for analyzing the sequences of other biological molecules, particularly those useful for comparing sequences when one sequence is known and the other is not.

As used herein the specification, “a” or “an” may mean one or more. As used herein in the claim(s), when used in conjunction with the word “comprising”, the words “a” or “an” may mean one or more than one. As used herein “another” may mean at least a second or more.

One skilled in the art recognizes that when first substrate and second substrate are referenced herein that both the first and second substrates could be different substrates or that a single substrate is used in both cases. In the later case, after use of the substrate as the first substrate, the conditions on the substrate are changed such that the sequences hybridized on the first use are removed and the substrate is then used as the second substrate.

All patents and publications mentioned in the specification are indicative of the level of those skilled in the art to which the invention pertains. All patents and publications are herein incorporated by reference to the same extent as if each individual publication was specifically and individually indicated to be incorporated by reference.

Definitions

“NSEQ” refers generally to a polynucleotide sequence of the present invention, including SEQ ID NOs: 7, 13, and 17. “PSEQ” refers generally to a polypeptide sequence of the present invention, including SEQ ID NOs: 8, 14, and 18.

A “variant” refers to either a polynucleotide or a polypeptide whose sequence diverges from SEQ ID NOs: 7, 13, or 17 or SEQ ID NOs: 8, 14, or 18, respectively. Polynucleotide sequence divergence may result from mutational changes such as deletions, additions, and substitutions of one or more nucleotides; it may also occur because of differences in codon usage. Each of these types of changes may occur alone, or in combination, one or more times in a given sequence. Polypeptide variants include sequences that possess at least one structural or functional characteristic of SEQ ID NOs: 8, 14, or 18.

“Gene” or “gene sequence” refers to the partial or complete coding sequence of a gene. The term also refers to 5′ or 3′ untranslated regions. The gene may be in a sense or antisense (complementary) orientation.

“Disease-specific gene” refers to a gene sequence which has been previously identified as useful in the diagnosis, treatment, prognosis, or prevention of a disease, and more preferably, in the diagnosis, treatment, prognosis, or prevention of cancer.

“Disease-associated gene” refers to a gene sequence whose expression pattern is similar to that of the disease-specific genes and which are useful in the diagnosis, treatment, prognosis, or prevention of disease. The gene sequences can also be used in the evaluation of therapies for disease.

“Substantially purified” refers to a nucleic acid or an amino acid sequence that is removed from its natural environment and is isolated or separated, and is at least about 60% free, preferably about 75% free, and most preferably about 90% free from other components with which it is naturally present.

The Invention

The present invention encompasses a method for identifying biomolecules that are associated with a specific disease, regulatory pathway, subcellular compartment, cell type, tissue type, or species. In particular, the method identifies gene sequences useful in diagnosis, prognosis, treatment, prevention, and evaluation of therapies for various diseases.

The method entails first identifying polynucleotides (or mRNAs) that are expressed in a biological system of interest. The polynucleotides include genes of known function, genes known to be specifically expressed in a specific disease process, subcellular compartment, cell type, tissue type, or species. Additionally, the polynucleotides include genes of unknown function. The expression patterns of the known genes are then compared with those of the genes of unknown function to determine whether a specified probability threshold is met. Through this comparison, a subset of the polynucleotides having a high probability of being co-differentially expressed with the known genes can be identified. The high probability correlates with a particular probability threshold which is less than 10⁻⁷, and more preferably less than 10⁻⁹.

The Microarrays

The polynucleotides that are deposited as targets on the microarrays originate from cDNA libraries derived from a variety of sources including, but not limited to, eukaryotes such as human, mouse, rat, dog, monkey, plant, and yeast and prokaryotes such as bacteria and viruses. These polynucleotides can also be selected from a variety of sequence types including, but not limited to, expressed sequence tags (ESTs), assembled polynucleotide sequences, full length gene coding regions, introns, regulatory sequences, 5′ untranslated regions, and 3′ untranslated regions.

The microarrays comprise polynucleotides from cDNA libraries obtained from blood vessels, heart, blood cells, cultured cells, connective tissue, epithelium, islets of Langerhans, neurons, phagocytes, biliary tract, esophagus, gastrointestinal system, liver, pancreas, fetus, placenta, chromaffin system, endocrine glands, ovary, uterus, penis, prostate, seminal vesicles, testis, bone marrow, immune system, cartilage, muscles, skeleton, central nervous system, ganglia, neuroglia, neurosecretory system, peripheral nervous system, bronchus, larynx, lung, nose, pleurus, ear, eye, mouth, pharynx, exocrine glands, bladder, kidney, ureter, and the like.

In a preferred embodiment, gene sequences are assembled to reflect related sequences, such as assembled sequence fragments derived from a single transcript. Assembly of the polynucleotide sequences can be performed using sequences of various types including, but not limited to, ESTs, extensions, or shotgun sequences. In a most preferred embodiment, the polynucleotide sequences are derived from human sequences that have been assembled using the algorithm disclosed in “Database and System for Storing, Comparing and Displaying Related Biomolecular Sequence Information”, Lincoln et al., Serial No. 60/079,469, filed Mar. 26, 1998, herein incorporated by reference.

Evaluation of Differential Expression

Experimentally, differential expression of the polynucleotides can be evaluated by methods including, but not limited to, differential display by spatial immobilization or by gel electrophoresis, genome mismatch scanning, representational difference analysis, and transcript imaging. Additionally, differential expression can be assessed by microarray technology. These methods may be used alone or in combination.

Preferably, a microarray is created by arraying individual polynucleotides on a substrate with each gene occupying a unique location. Differential expression is assessed by dual channel microarray technology. More specifically, samples of mRNA from treated cells are purified, fluorescently labeled, and competitively hybridized against an untreated reference sample labeled with a different fluorochrome. After hybridization and washing, the microarrays are scanned for the two different fluorescent labels.

Image-processing algorithms calculate the signal generated from each fluorescent probe on each element. More specifically, it has been found that the ratio of the two fluorescent intensities provides a highly accurate and quantitative measurement of the relative gene expression level in the two cell samples. For example, if a microarray element shows no fluorescence, it indicates that the gene in that element was not expressed in either cell sample. If an element shows a single color, it indicates that a labeled gene was expressed only in that cell sample. The appearance of both colors indicates that the gene was expressed in both cell samples. Even genes expressed once per cell (1 part in 100,000 sensitivity) can be detected using this technology. Two-fold or more changes of expression intensity are also readily detectable. Expression ratios can be calculated for those elements with sufficient signal in at least one channel.

The number of microarray images used in the analyses can range from as few as 20 to greater than 10,000. Preferably, the number of the dual channel microarray images used in the analyses described herein for estimating the probability that two polynucleotides are co-differentially expressed is greater than 200.

Statistical Analysis of Co-Differential Expression

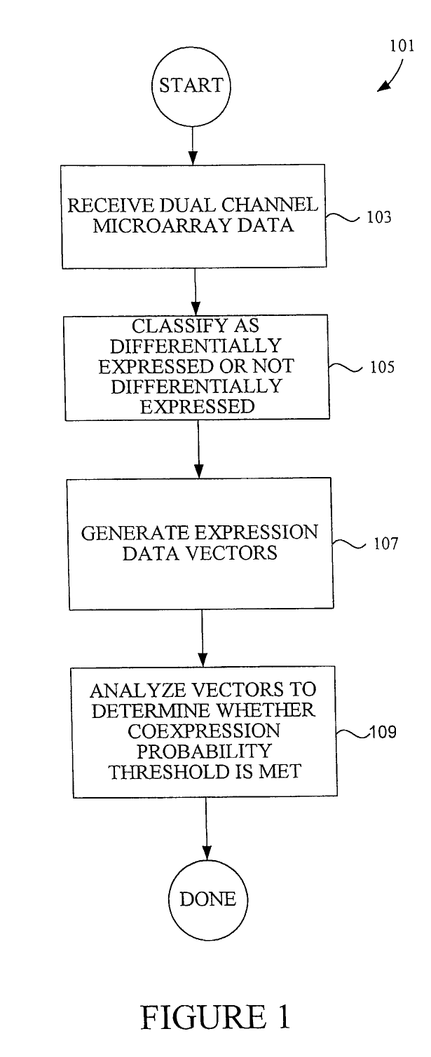

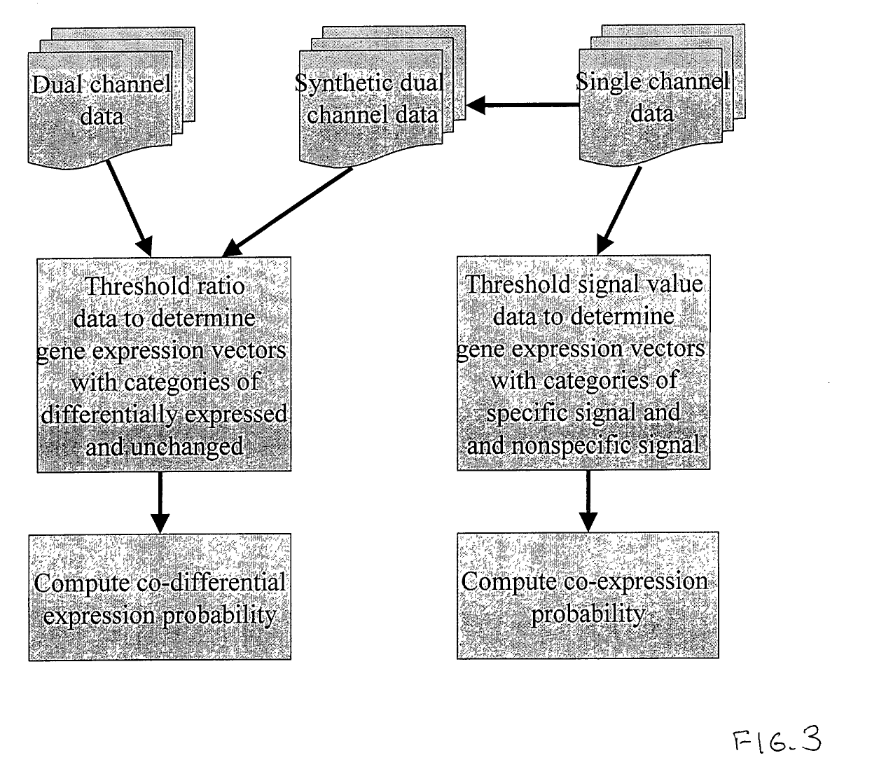

A high level process flow 101 in accordance with one embodiment of this invention for identifying novel genes that exhibit a statistically significant co-differential expression pattern with a target gene is depicted in FIG. 1. See also, FIG. 3. The process begins at 103 with the dual channel microarray data. The data can be obtained directly using dual channel technology as described above. In one embodiment, synthetic dual channel data is created by obtaining single channel data and taking ratios between different microarray experiments.

At 105, each gene sequence is then classified as either being differentially expressed or as not being differentially expressed. This determination may require a properly selected threshold for differential expression. In practice, a useful selection of this threshold can be done empirically using techniques known in the art and is done commonly. See, e.g., U.S. Pat. No. 6,245,517, which is incorporated herein by reference.

Once the microarray data has been classified into the mutually exclusive categories of differentially expressed and not differentially expressed, statistical analysis can be performed to determine whether two genes are co-differentially expressed.

To determine whether two genes, A and B, have similar differential expression patterns, at 107, expression data vectors can be generated as illustrated in Table 1, wherein a differentially expressed gene is indicated by a one and a non-differentially expressed gene by a zero. In other words, a “one” indicates that a gene is differentially expressed at a ratio that is greater than the threshold (e.g., +/−2 fold) and a “zero” indicates that a gene is not differentially expressed (e.g., shows less than a +/−2 fold change in expression between treated and untreated samples).

TABLE 1
Expression data vectors for genes A and B

| | Microarray | Microarray | Microarray | | Microarray |
|---|---|---|---|---|---|
| | Hybridiza- | Hybridiza- | Hybridiza- | | Hybridization |
| | tion 1 | tion 2 | tion 3 | . . . | N |
| gene A | 1 | 1 | 0 | . . . | 0 |
| gene B | 1 | 0 | 1 | . . . | 0 |

For a given pair of genes, the expression data vectors are summarized in a 2×2 contingency table.

TABLE 2
Contingency table for co-differential expression of genes A and B

| | Gene A | Gene A | |
|---|---|---|---|
| | 2-fold +/− | No change | Total |
| Gene B | 8 | 2 | 10 |
| 2-fold +/− | | | |
| Gene B | 2 | 18 | 20 |
| No change | | | |
| Total | 10 | 20 | 30 |

Table 2 presents co-differential expression data for gene A and gene B in a total of 30 libraries. Table 2 summarizes and presents 1) the number of times gene A and B both display a 2-fold increase or decrease, 2) the number of times gene A and B both show no change in expression; 3) the number of times gene A shows a 2-fold increase or decrease in expression while gene B shows no change, and 4) the number of times gene B shows a 2-fold increase or decrease in expression while gene A shows no change. The upper left entry is the number of times the two genes are differentially expressed, and the middle right entry is the number of times neither gene is differentially expressed. The off diagonal entries are the number of times one gene is differentially expressed while the other does not.

The vectors are then analyzed at 109 to determine whether the expression patterns of any of the genes are similar. Expression patterns are similar if a particular probability threshold is met. The significance of gene co-differential expression is evaluated using a probability method to measure a due-to-chance probability of the co-differential expression. The probability method can be the Fisher exact test, the chi-squared test, or the kappa test. These tests and examples of their applications are well known in the art and can be found in standard statistics texts (Agresti, A. (1990) Categorical Data Analysis. New York, N.Y., Wiley; Rice, J. A. (1988) Mathematical Statistics and Data Analysis. Pacific Grove, Calif., Wadsworth & Brooks/Cole). A Bonferroni correction (Rice, supra, page 384) can also be applied in combination with one of the probability methods for correcting statistical results of one gene versus multiple other genes.

This method of estimating the probability for co-differential expression of two genes makes several assumptions. The method assumes that the libraries are independent and are identically sampled. However, in practical situations, the selected cDNA libraries are not entirely independent because more than one library may be obtained from a single patient or tissue, and they are not entirely identically sampled because different numbers of cDNA's may be sequenced from each library (typically ranging from 5,000 to 10,000 cDNA's per library). In addition, because a Fisher exact probability is calculated for each gene versus 41,419 other genes, a Bonferroni correction for multiple statistical tests is necessary.

The probability (“p-value”) that the simultaneous 2-fold change in expression for gene A and gene B occurs due to chance as calculated using a Fisher exact test is 0.0003. In a preferred embodiment, the due-to-chance probability is measured by a Fisher exact test, and the threshold of the due-to-chance probability is set to less than 10⁻⁷, more preferably less than 10⁻⁹.

Evaluation of Co-Expression

Microarray-based experiments are presently a preferred method to generate gene expression data. Microarrays consist of an ordered arrangement of known gene sequences, or array elements, immobilized on a substrate. To generate gene expression data, the array elements are probed with a sample. The sample may have been derived, for example, from tissue of an individual suffering from a disease, from tissue treated in a specified manner or a control tissue. Samples are typically prepared by isolating mRNA, or its equivalent, and then labeling the mRNA with a fluorescent reporter group. The labeled mRNA sample is then combined with microarray array elements to form hybridization complexes between array elements and mRNA molecules that have identical or similar sequences (complementary sequences). Those labeled mRNA molecules that do not have a sequence complementary to the array element sequences are removed by a series of washes. Any formed complexes are detected by using a scanner to measure fluorescent signals emitted from specific locations on the microarray. Since the position and sequence of each array element is known, microarrays are an effective way to determine which specific genes are expressed in a sample.

The microarray hybridization experiments may be performed using one of several formats. In one format, a microarray is probed using a single labeled mRNA sample and what is detected after complex formation is a measurement of levels of particular mRNAs in a sample. Image-processing algorithms calculate the signal generated from each fluorescent probe on each element. Even genes expressed once per cell (1 part in 100,000 sensitivity) can be detected using this technology.

The number of microarray images used in the analyses can range from as few as 20 to greater than 10,000. Preferably, the number of the microarray images used in the analyses described herein for estimating the probability that two polynucleotides are co-expressed is greater than 200.

Statistical Analysis of Co-Expression

In another embodiment of the invention, single channel data is used directly to determine co-expression of two genes. Each gene sequence is first classified as either being specific signal or as being nonspecific signal using a threshold signal value. See, FIG. 3.

A threshold for single channel data can be defined by various approaches. One method is to estimate the distribution of signal values for negative controls by using explicit negative controls on the microarray. One can also estimate this distribution by assuming that most genes are not expressed at significant levels in any given sample and use the distribution of the lower 70% to 90% of the signals as an approximation. The variance of this distribution should also be estimated and used to define a threshold above which a sufficiently small number of false positives would come from the negative control distribution. So there would be reasonable confidence that signals above this level are specific. Other measures of nonspecific signals such as cross hybridization analysis by sequence similarity could also be used to increase confidence in whether the signal is specific for the gene of interest although ideally this would be taken into account during microarray design. Although Pearson's and Spearman's may work well for many or most cases, a categorical method as described herein can detect nonlinear relationships missed by these methods and thus be an important complementary method of analysis.

Once the microarray data has been classified into the mutually exclusive categories of specific signal and nonspecific signal, statistical analysis can be performed, as described above, to determine whether two genes are co-expressed.

### EXAMPLES

Using the method of the present invention, five genes have been identified that exhibit strong association, or co-differential expression, with a known gene, human tumor necrosis factor alpha inducible protein A20. The results presented in Table 3 show that the expression of five genes, one of which is novel, have direct or indirect association with the expression of A20. Therefore, this novel gene can be used in the diagnosis, treatment, prognosis, or prevention of cancer, or in the evaluation of therapies for cancer. Further, the gene product of the novel gene is a potential therapeutic protein and target of anti-cancer therapeutics.

TABLE 3
Co-differential Expression Analysis with Protein A20

| | Genbank | | |
|---|---|---|---|
| P-value | Identifier | Description | Role |
| 2.9e-120 | G177865 | Human tumor necrosis factor | Blocks TNF-in- |
| | | alpha inducible protein A20 | duced apoptosis. |
| | | (SEQ ID NOs: 1 and 2) | Induced by TNF. |
| | | | Inhibitor of |
| | | | NF-kappaB. |
| 3.0e-37 | G183628 | Human cytokine (GRO-beta) | Chemotactic for |
| | | mRNA (SEQ ID NOs: 3 and | neutorphilic |
| | | 4) | granulocytes. |
| | | | Binds IL-8R. In- |
| | | | duced by TNF. |
| 6.4e-36 | G179579 | Human IL-8 (SEQ ID NOs: 5 | Activates neutro- |
| | | and 6) | phil granulocytes. |
| | | | Induced by TNF. |
| 1.9e-34 | Not | SEQ ID NO: 7 and SEQ ID | TNF-inducible |
| | applicable | N0: 8 | chemokine. |
| 4.9e-34 | G183622 | Human GRO (growth regu- | Neutrophil |
| | | lated) gene (SEQ ID NOs: 9 | chemoattractant. |
| | | and 10) | Binds IL-8R. In- |
| | | | duced by TNF. |
| 4.3e-25 | G1694788 | Human mRNA for GRS pro- | Blocks apoptosis |
| | | tein (SEQ ID NOs: 11 and 12) | by TNF, p53. In- |
| | | | duced by TNF. |

Therefore, in one embodiment, the present invention encompasses a polynucleotide sequence comprising the sequence of SEQ ID NO:7. This polynucleotide has been shown by the method of the present invention to have strong association (or high probability for being co-differentially expressed) with a variety of TNF-inducible chemokines. The invention also encompasses a variant of the polynucleotide sequence and its complement. Variant polynucleotide sequences typically have at least about 70%, more preferably at least about 85%, and most preferably at least about 95% polynucleotide sequence identity to SEQ ID NO:7.

Using the method of the present invention, eight genes that exhibit strong association, or co-differential expression, with a novel gene, SEQ ID NO:13, have been identified. The results presented in Table 4 show that the expression of eight genes, one of which is novel, have direct or indirect association with the SEQ ID NO:13.

TABLE 4
Co-differential Expression Analysis with Novel Gene SEQ ID NO: 13

| | Genbank | |
|---|---|---|
| P-value | Identifier | Description |
| 3.5e-32 | Not | SEQ ID NOs: 13 and 14 |
| | applicable | |
| 3.2e-16 | G5726288 | Human calcim-activaated chloride channel (SEQ |
| | | ID NOs: 15 and 16) |
| 2.5e-11 | Not | SEQ ID NOs: 17 and 18 |
| | applicable | |
| 5.7e-11 | G291963 | Human colon mucosa-associated (DRA) mRNA |
| | | (SEQ ID NOs: 19 and 20) |
| 5.7e-11 | G183414 | Human guanylin mRNA, complete cds. (SEQ ID |
| | | NOs: 21 and 22) |
| 1.2e-10 | G179792 | Human carbonic anhydrase I (CAI) (SEQ ID NOs: |
| | | 23 and 24) |
| 1.6e-10 | G409457 | Human calcium-dependent chloride channel (SEQ |
| | | ID NOs: 25 and 26) |
| 1.6e-10 | G4753765 | Human mRNA for UDP-glucuronosyltransferase |
| | | (UGT) (SEQ ID NOs: 27 and 28) |
| 4.4e-10 | G2385453 | Human mRNA for galectin-4 (SEQ ID NOs: 29 and |
| | | 30) |

Inspection of these results reveals that the majority of the genes are digestive tract/colon specific. In addition, three of the genes are associated with adenocarcinoma, including DRA or “Down Regulated in Adenoma”. Chloride channel genes have also been associated with colon cancer, although these changes may be a side effect of the cancer rather than a mechanism of the cancer. It has also been shown that uroguanylin treatment suppresses polyp formation and induces apoptosis in human colon adenocarcinoma cells. As such, the analysis indicates that SEQ ID NO:13 and SEQ ID NO:17 may be involved with cancer of the digestive tract and/or colon. Therefore, these two novel genes can potentially be used in diagnosis, treatment, prognosis, or prevention of cancer, or in the evaluation of therapies for cancer. Further, the gene products of these two genes are potential therapeutic proteins and targets of anti-cancer therapeutics.

Therefore, in one embodiment, the present invention encompasses a polynucleotide sequence comprising the sequence of SEQ ID NO:13 or SEQ ID NO:17. The invention also encompasses a variant of the polynucleotide sequence and its complement. Variant polynucleotide sequences typically have at least about 70%, more preferably at least about 85%, and most preferably at least about 95% polynucleotide sequence identity to SEQ ID NO:13 or SEQ ID NO:17.

One preferred method for identifying variants entails using NSEQ and/or PSEQ sequences to search against the GenBank primate (pri), rodent (rod), and mammalian (mam), vertebrate (vrtp), and eukaryote (eukp) databases, SwissProt, BLOCKS (Bairoch, A. et al. (1997) Nucleic Acids Res. 25:217-221), PFAM, and other databases that contain previously identified and annotated motifs, sequences, and gene functions. Methods that search for primary sequence patterns with secondary structure gap penalties (Smith, T. et al. (1992) Protein Engineering 5:35-51) as well as algorithms such as BLAST (Basic Local Alignment Search Tool; Altschul, S. F. (1993) J. Mol. Evol 36:290-300; and Altschul et al. (1990) J. Mol. Biol. 215:403-410), BLOCKS (Henikoff S. and Henikoff G. J. (1991) Nucleic Acids Research 19:6565-6572), Hidden Markov Models (HMM; Eddy, S. R. (1996) Cur. Opin. Str. Biol. 6:361-365; and Sonnhammer, E. L. L. et al. (1997) Proteins 28:405-420), and the like, can be used to manipulate and analyze nucleotide and amino acid sequences. These databases, algorithms and other methods are well known in the art and are described in Ausubel, F. M. et al. (1997; Short Protocols in Molecular Biology, John Wiley & Sons, New York, N.Y.) and in Meyers, R. A. (1995; Molecular Biology and Biotechnology, Wiley V C H, Inc, New York, N.Y., p 856-853).

Also encompassed by the invention are polynucleotide sequences that are capable of hybridizing to SEQ ID NO: 7, SEQ ID NO:13, and SEQ ID NO:17, and fragments thereof under stringent conditions. Stringent conditions can be defined by salt concentration, temperature, and other chemicals and conditions well known in the art. In particular, stringency can be increased by reducing the concentration of salt, or raising the hybridization temperature.

For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Stringent temperature conditions will ordinarily include temperatures of at least about 30@C, more preferably of at least about 37@C, and most preferably of at least about 42@C. Varying additional parameters, such as hybridization time, the concentration of detergent (sodium dodecyl sulfate, SDS) or solvent (formamide), and the inclusion or exclusion of carrier DNA, are well known to those skilled in the art. Additional variations on these conditions will be readily apparent to those skilled in the art (Wahl, G. M. and S. L. Berger (1987) Methods Enzymol. 152:399-407; Kimmel, A. R. (1987) Methods Enzymol. 152:507-511; Ausubel, F. M. et al. (1997) Short Protocols in Molecular Biology, John Wiley & Sons, New York, N.Y.; and Sambrook, J. et al. (1989) Molecular Cloning, A Laboratory Manual, Cold Spring Harbor Press, Plainview, N.Y.).

NSEQ or the polynucleotide sequences encoding PSEQ can be extended utilizing a partial nucleotide sequence and employing various PCR-based methods known in the art to detect upstream sequences, such as promoters and regulatory elements. (See, e.g., Dieffenbach, C. W. and G. S. Dveksler (1995; PCR Primer, a Laboratory Manual, Cold Spring Harbor Press, Plainview, N.Y., pp.1-5; Sarkar, G. (1993; PCR Methods Applic. 2:318-322); Triglia, T. et al. (1988; Nucleic Acids Res. 16:8186); Lagerstrom, M. et al. (1991; PCR Methods Applic. 1:111-119); and Parker, J. D. et al. (1991; Nucleic Acids Res. 19:3055-306). Additionally, one may use PCR, nested primers, and PROMOTERFINDER libraries to walk genomic DNA (Clontech, Palo Alto, Calif.). This procedure avoids the need to screen libraries and is useful in finding intron/exon junctions. For all PCR-based methods, primers may be designed using commercially available software, such as OLIGO 4.06 Primer Analysis software (National Biosciences Inc., Plymouth Minn.) or another appropriate program, to be about 18 to 30 nucleotides in length, to have a GC content of about 50% or more, and to anneal to the template at temperatures of about 68@C to 72@C.

In another aspect of the invention, NSEQ or the polynucleotide sequences encoding PSEQ can be cloned in recombinant DNA molecules that direct expression of PSEQ or the polypeptides encoded by NSEQ, or structural or functional fragments thereof, in appropriate host cells. Due to the inherent degeneracy of the genetic code, other DNA sequences which encode substantially the same or a functionally equivalent amino acid sequence may be produced and used to express the polypeptides of PSEQ or the polypeptides encoded by NSEQ. The nucleotide sequences of the present invention can be engineered using methods generally known in the art in order to alter the nucletide sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

In order to express a biologically active polypeptide encoded by NSEQ, NSEQ or the polynucleotide sequences encoding PSEQ, or derivatives thereof, may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in NSEQ or polynucleotide sequences encoding PSEQ. Methods which are well known to those skilled in the art may be used to construct expression vectors containing NSEQ or polynucleotide sequences encoding PSEQ and appropriate transcriptional and translational control elements. These methods include in vitro recombinant DNA techniques, synthetic techniques, and in vivo genetic recombination. (See, e.g., Sambrook (supra) and Ausubel, (supra).

A variety of expression vector/host cell systems may be utilized to contain and express NSEQ or polynucleotide sequences encoding PSEQ. These include, but are not limited to, microorganisms such as bacteria transformed with recombinant bacteriophage, plasmid, or cosmid DNA expression vectors; yeast transformed with yeast expression vectors; insect cell systems infected with viral expression vectors (baculovirus); plant cell systems transformed with viral expression vectors, cauliflower mosaic virus (CaMV) or tobacco mosaic virus (TMV), or with bacterial expression vectors (Ti or pBR322 plasmids); or animal cell systems. The invention is not limited by the host cell employed. For long term production of recombinant proteins in mammalian systems, stable expression of a polypeptide encoded by NSEQ in cell lines is preferred. For example, NSEQ or sequences encoding PSEQ can be transformed into cell lines using expression vectors which may contain viral origins of replication and/or endogenous expression elements and a selectable marker gene on the same or on a separate vector.

In general, host cells that contain NSEQ and that express PSEQ may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences. Immunological methods for detecting and measuring the expression of PSEQ using either specific polyclonal or monoclonal antibodies are known in the art. Examples of such techniques include enzyme-linked immunosorbent assays (ELISAs), radioimmunoassays (RIAs), and fluorescence activated cell sorting (FACS).

Host cells transformed with NSEQ or polynucleotide sequences encoding PSEQ may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides of NSEQ or polynucleotides encoding PSEQ may be designed to contain signal sequences which direct secretion of PSEQ or polypeptides encoded by NSEQ through a prokaryotic or eukaryotic cell membrane.

In addition, a host cell strain may be chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to specify protein targeting, folding, and/or activity. Different host cells which have specific cellular machinery and characteristic mechanisms for post-translational activities (e.g., CHO, HeLa, MDCK, HEK293, and W138), are available from the American Type Culture Collection (ATCC, Bethesda, Md.) and may be chosen to ensure the correct modification and processing of the foreign protein.

In another embodiment of the invention, natural, modified, or recombinant NSEQ or nucleic acid sequences encoding PSEQ are ligated to a heterologous sequence resulting in translation of a fusion protein containing heterologous protein moieties in any of the aforementioned host systems. Such heterologous protein moieties facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, hemagglutinin (HA) and monoclonal antibody epitopes.

In another embodiment, NSEQ or sequences encoding PSEQ are synthesized, in whole or in part, using chemical methods well known in the art. (See, e.g., Caruthers, M. H. et al. (1980) Nucl. Acids Res. Symp. Ser. 215-223; Horn, T. et al. (1980) Nucl. Acids Res. Symp. Ser. 225-232; and Ausubel, supra). Alternatively, PSEQ or a polypeptide sequence encoded by NSEQ itself, or a fragment thereof, may be synthesized using chemical methods. For example, peptide synthesis can be performed using various solid-phase techniques (Roberge, J. Y. et al. (1995) Science 269:202-204). Automated synthesis may be achieved using the ABI 431A Peptide Synthesizer (Perkin Elmer). Additionally, PSEQ or the amino acid sequence encoded by NSEQ, or any part thereof, may be altered during direct synthesis and/or combined with sequences from other proteins, or any part thereof, to produce a polypeptide variant.

In another embodiment, the invention entails a substantially purified polypeptide comprising the amino acid sequence selected from the group consisting of SEQ ID NO:8, SEQ ID NO:14, SEQ ID NO:18, or fragments thereof. SEQ ID NO:8 is encoded by SEQ ID NO:7 and is a potential TNF-inducible chemokine. SEQ ID NO:18 and SEQ ID NO:14 are encoded by SEQ ID NO:17 and SEQ ID NO:13, respectively and may be involved with cancer of the digestive tract and/or colon.

Diagnostics and Therapeutics

The sequences of the these genes can be used in diagnosis, prognosis, treatment, prevention, and evaluation of therapies for diseases associated with cell proliferation, particularly cancer. Further, the amino acid sequences encoded by the novel genes are potential therapeutic proteins and targets of anti-cancer therapeutics.

In one preferred embodiment, the polynucleotide sequences of NSEQ or the polynucleotides encoding PSEQ are used for diagnostic purposes to determine the absence, presence, and excess expression of PSEQ, and to monitor regulation of the levels of mRNA or the polypeptides encoded by NSEQ during therapeutic intervention. The polynucleotides may be at least 18 nucleotides long, complementary RNA and DNA molecules, branched nucleic acids, and peptide nucleic acids (PNAs). Alternatively, the polynucleotides are used to detect and quantitate gene expression in samples in which expression of PSEQ or the polypeptides encoded by NSEQ are correlated with disease. Additionally, NSEQ or the polynucleotides encoding PSEQ can be used to detect genetic polymorphisms associated with a disease. These polymorphisms may be detected at the transcript cDNA or genomic level.

The specificity of the probe, whether it is made from a highly specific region, e.g., the 5′ regulatory region, or from a less specific region, e.g., a conserved motif, and the stringency of the hybridization or amplification (maximal, high, intermediate, or low), will determine whether the probe identifies only naturally occurring sequences encoding PSEQ, allelic variants, or related sequences.

Probes may also be used for the detection of related sequences, and should preferably have at least 50% sequence identity to any of the NSEQ or PSEQ-encoding sequences.

Means for producing specific hybridization probes for DNAs encoding PSEQ include the cloning of NSEQ or polynucleotide sequences encoding PSEQ into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, by fluorescent labels and the like. The polynucleotide sequences encoding PSEQ may be used in Southern or northern analysis, dot blot, or other membrane-based technologies; in PCR technologies; and in microarrays utilizing fluids or tissues from patients to detect altered PSEQ expression. Such qualitative or quantitative methods are well known in the art.

NSEQ or the nucleotide sequences encoding PSEQ can be labeled by standard methods and added to a fluid or tissue sample from a patient under conditions suitable for the formation of hybridization complexes. After a suitable incubation period, the sample is washed and the signal is quantitated and compared with a standard value. If the amount of signal in the patient sample is significantly altered in comparison to the standard value then the presence of altered levels of nucleotide sequences of NSEQ and those encoding PSEQ in the sample indicates the presence of the associated disease. Such assays may also be used to evaluate the efficacy of a particular therapeutic treatment regimen in animal studies, in clinical trials, or to monitor the treatment of an individual patient.

Once the presence of a disease is established and a treatment protocol is initiated, hybridization or amplification assays can be repeated on a regular basis to determine if the level of expression in the patient begins to approximate that which is observed in the normal subject. The results obtained from successive assays may be used to show the efficacy of treatment over a period ranging from several days to months.

The polynucleotides may be used for the diagnosis of a variety of diseases associated with cell proliferation including cancer such as adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus.

Alternatively, the polynucleotides may be used as targets in a microarray. The microarray can be used to monitor the expression level of large numbers of genes simultaneously and to identify splice variants, mutations, and polymorphisms. This information may be used to determine gene function, to understand the genetic basis of a disease, to diagnose a disease, and to develop and monitor the activities of therapeutic agents.

In yet another alternative, polynucleotides may be used to generate hybridization probes useful in mapping the naturally occurring genomic sequence. Fluorescent in situ hybridization (FISH) may be correlated with other physical chromosome mapping techniques and genetic map data. (See, e.g., Heinz-Ulrich, et al. (1995) in Meyers, R. A. (ed.) Molecular Biology and Biotechnology, VCH Publishers New York, N.Y., pp. 965-968).

In another embodiment, antibodies which specifically bind PSEQ may be used for the diagnosis of diseases characterized by the over-or-underexpression of PSEQ or polypeptides encoded by NSEQ. Alternatively, one may use competitive drug screening assays in which neutralizing antibodies capable of binding PSEQ or the polypeptides encoded by NSEQ specifically compete with a test compound for binding the polypeptides. In this manner, antibodies can be used to detect the presence of any peptide which shares one or more antigenic determinants with PSEQ or the polypeptides encoded by NSEQ. Diagnostic assays for PSEQ or the polypeptides encoded by NSEQ include methods which utilize the antibody and a label to detect PSEQ or the polypeptided encoded by NSEQ in human body fluids or in extracts of cells or tissues. A variety of protocols for measuring PSEQ or the polypeptides encoded by NSEQ, including ELISAs, RIAs, and FACS, are well known in the art and provide a basis for diagnosing altered or abnormal levels of the expression of PSEQ or the polypeptides encoded by NSEQ. Normal or standard values for PSEQ expression are established by combining body fluids or cell extracts taken from normal subjects, preferably human, with antibody to PSEQ or a polypeptide encoded by NSEQ under conditions suitable for complex formation The amount of standard complex formation may be quantitated by various methods, preferably by photometric means. Quantities of PSEQ or the polypeptides encoded by NSEQ expressed in subject, control, and disease samples from biopsied tissues are compared with the standard values. Deviation between standard and subject values establishes the parameters for diagnosing or monitoring disease.

In another aspect, the polynucleotides and polypeptides of the present invention can be employed for treatment or the monitoring of therapeutic treatments for cancers. The polynucleotides of NSEQ or those encoding PSEQ, or any fragment or complement thereof, may be used for therapeutic purposes. In one aspect, the complement of the polynucleotides of NSEQ or those encoding PSEQ may be used in situations in which it would be desirable to block the transcription or translation of the mRNA.

Expression vectors derived from retroviruses, adenoviruses, or herpes or vaccinia viruses, or from various bacterial plasmids, may be used for delivery of nucleotide sequences to the targeted organ, tissue, or cell population. Methods which are well known to those skilled in the art can be used to construct vectors to express nucleic acid sequences complementary to the polynucleotides encoding PSEQ. (See, e.g., Sambrook, supra; and Ausubel, supra.)

Genes having polynucleotide sequences of NSEQ or those encoding PSEQ can be turned off by transforming a cell or tissue with expression vectors which express high levels of a polynucleotide, or fragment thereof, encoding PSEQ. Such constructs may be used to introduce untranslatable sense or antisense sequences into a cell. Oligonucleotides derived from the transcription initiation site, e.g., between about positions −10 and +10 from the start site, are preferred. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee, J. E. et al. (1994) in Huber, B. E. and B. I. Carr, Molecular and Immunologic Approaches, Futura Publishing Co., Mt. Kisco, N.Y., pp. 163-177.) Ribozymes, enzymatic RNA molecules, may also be used to catalyze the specific cleavage of RNA.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art. (See, e.g., Goldman, C. K. et al. (1997) Nature Biotechnology 15:462-466.)

Further, an antagonist or antibody of a polypeptide of PSEQ or encoded by NSEQ may be administered to a subject to treat or prevent a cancer associated with increased expression or activity of PSEQ. An antibody which specifically binds the polypeptide may be used directly as an antagonist or indirectly as a targeting or delivery mechanism for bringing a pharmaceutical agent to cells or tissue which express the the polypeptide.

Antibodies to PSEQ or polypeptides encoded by NSEQ may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are especially preferred for therapeutic use. Monoclonal antibodies to PSEQ may be prepared using any technique which provides for the production of antibody molecules by continuous cell lines in culture. These include, but are not limited to, the hybridoma technique, the human B-cell hybridoma technique, and the EBV-hybridoma technique. In addition, techniques developed for the production of chimeric antibodies can be used. (See, for example, Molecular Biology and Biotechnology, R. A. Myers, ed., (1995) John Wiley & Sons, Inc., New York, N.Y.). Alternatively, techniques described for the production of single chain antibodies may be employed. Antibody fragments which contain specific binding sites for PSEQ or the polypeptide sequences encoded by NSEQ may also be generated.

Various immunoassays may be used for screening to identify antibodies having the desired specificity. Numerous protocols for competitive binding or immunoradiometric assays using either polyclonal or monoclonal antibodies with established specificities are well known in the art.

Yet further, an agonist of a polypeptide of PSEQ or that encoded by NSEQ may be administered to a subject to treat or prevent a cancer associated with decreased expression or activity of the polypeptide.

An additional aspect of the invention relates to the administration of a pharmaceutical or sterile composition, in conjunction with a pharmaceutically acceptable carrier, for any of the therapeutic effects discussed above. Such pharmaceutical compositions may consist of polypeptides of PSEQ or those encoded by NSEQ, antibodies to the polypeptides, and mimetics, agonists, antagonists, or inhibitors of the polypeptides. The compositions may be administered alone or in combination with at least one other agent, such as a stabilizing compound, which may be administered in any sterile, biocompatible pharmaceutical carrier including, but not limited to, saline, buffered saline, dextrose, and water. The compositions may be administered to a patient alone, or in combination with other agents, drugs, or hormones.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

For any compound, the therapeutically effective dose can be estimated initially either in cell culture assays, e.g., of neoplastic cells or in animal models such as mice, rats, rabbits, dogs, or pigs. An animal model may also be used to determine the appropriate concentration range and route of administration. Such information can then be used to determine useful doses and routes for administration in humans.

A therapeutically effective dose refers to that amount of active ingredient, for example, polypeptides of PSEQ or those encoded by NSEQ, or fragments thereof, antibodies of the polypeptides, and agonists, antagonists or inhibitors of the polypeptides, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics.

Any of the therapeutic methods described above may be applied to any subject in need of such therapy, including, for example, mammals such as dogs, cats, cows, horses, rabbits, monkeys, and most preferably, humans.

Apparatus

Generally, embodiments of the present invention employ various processes involving data stored in or transferred through one or more computer systems. Embodiments of the present invention also relate to an apparatus for performing these operations. This apparatus may be specially constructed for the required purposes, or it may be a general-purpose computer selectively activated or reconfigured by a computer program and/or data structure stored in the computer. The processes presented herein are not inherently related to any particular computer or other apparatus. In particular, various general-purpose machines may be used with programs written in accordance with the teachings herein, or it may be more convenient to construct a more specialized apparatus to perform the required method steps. A particular structure for a variety of these machines will appear from the description given below.

In addition, embodiments of the present invention relate to computer readable media or computer program products that include program instructions and/or data (including data structures) for performing various computer-implemented operations. Examples of computer-readable media include, but are not limited to, magnetic media such as hard disks, floppy disks, and magnetic tape; optical media such as CD-ROM disks; magneto-optical media; semiconductor memory devices, and hardware devices that are specially configured to store and perform program instructions, such as read-only memory devices (ROM) and random access memory (RAM). The data and program instructions of this invention may also be embodied on a carrier wave or other transport medium. Examples of program instructions include both machine code, such as produced by a compiler, and files containing higher level code that may be executed by the computer using an interpreter.

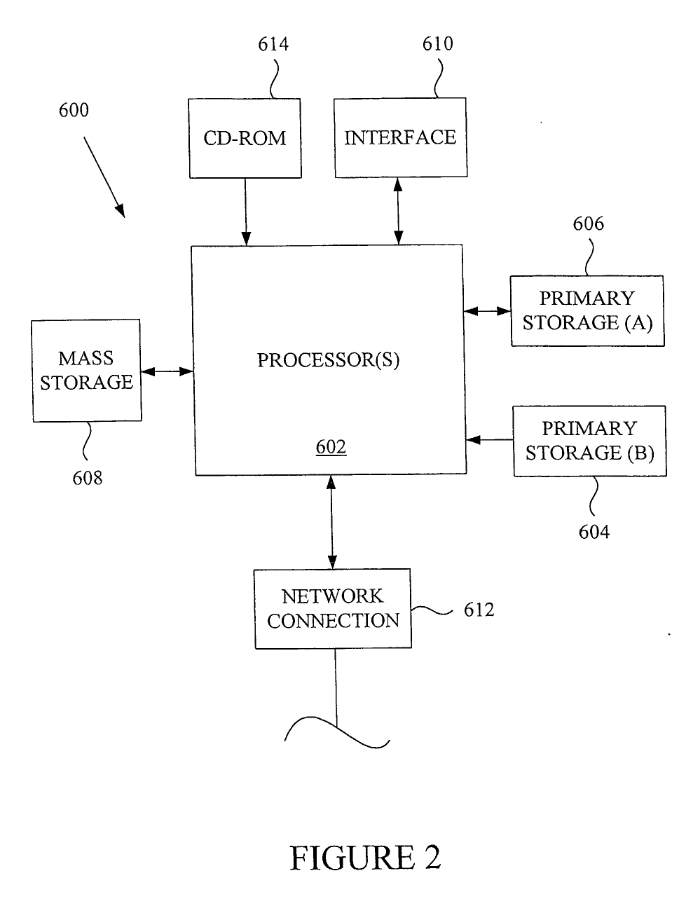

FIG. 2 illustrates a typical computer system that, when appropriately configured or designed, can serve as an image analysis apparatus of this invention. The computer system 600 includes any number of processors 602 (also referred to as central processing units, or CPUs) that are coupled to storage devices including primary storage 606 (typically a random access memory, or RAM), primary storage 604 (typically a read only memory, or ROM). CPU 602 may be of various types including microcontrollers and microprocessors such as programmable devices (e.g., CPLDs and FPGAs) and unprogrammable devices such as gate array ASICs or general purpose microprocessors. As is well known in the art, primary storage 604 acts to transfer data and instructions uni-directionally to the CPU and primary storage 606 is used typically to transfer data and instructions in a bi-directional manner. Both of these primary storage devices may include any suitable computer-readable media such as those described above. A mass storage device 608 is also coupled bi-directionally to CPU 602 and provides additional data storage capacity and may include any of the computer-readable media described above. Mass storage device 608 may be used to store programs, data and the like and is typically a secondary storage medium such as a hard disk. It will be appreciated that the information retained within the mass storage device 608, may, in appropriate cases, be incorporated in standard fashion as part of primary storage 606 as virtual memory. A specific mass storage device such as a CD-ROM 614 may also pass data uni-directionally to the CPU.

CPU 602 is also coupled to an interface 610 that connects to one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 602 optionally may be coupled to an external device such as a database or a computer or telecommunications network using an external connection as shown generally at 612. With such a connection, it is contemplated that the CPU might receive information from the network, or might output information to the network in the course of performing the method steps described herein.

In one embodiment, the computer system 600 is directly coupled to an electrophoresis detection instrument. Data from the electrophoresis detection instrument are provided via interface 612 for analysis by system 600. Alternatively, the data or traces processed by system 600 are provided from a data storage source such as a database or other repository. Again, the images are provided via interface 612. Once in the computer system 600, a memory device such as primary storage 606 or mass storage 608 buffers or stores, at least temporarily, the data or trace images. With this data, the image analysis apparatus 600 can perform various analysis operations such as statistical analyses. To this end, the processor may perform various operations on the stored images or data.

It is understood that this invention is not limited to the particular methodology, protocols, and reagents described, as these may vary. It is also understood that the terminology used herein is for the purpose of describing particular embodiments only, and is not intended to limit the scope of the present invention which will be limited only by the appended claims.

It is to be understood that the above description is intended to be illustrative and not restrictive. Many embodiments will be apparent to those skilled in the art upon reviewing the above description. The scope of the invention should, therefore, be determined not with reference to the above description, but should instead be determined with reference to the appended claims, along with the full scope of equivalents to which such claims are entitled.

What is claimed is:

1. A method for analyzing gene expression, the method comprising:
a) receiving a plurality of dual channel DNA microarray images;
b) analyzing said images to determine expression patterns of one or more disease-specific genes and one or more genes of unknown function; and
c) comparing the expression patterns of said disease-specific genes with the expression patterns of the genes of unknown function to identify a subset of the genes of unknown function which have similar expression patterns to those of the disease-specific genes.

2. The method of claim 1, wherein said obtaining dual channel DNA microarray images comprises
i) receiving a plurality of single channel DNA microarray images; and
ii) determining the ratio between said single channel DNA microarray images to yield a plurality of dual channel DNA microarray images.

3. The method of claim 1, wherein said comparing comprises
i) generating an expression data vector for each expressed gene by categorizing whether each gene is differential expressed or not differentially expressed;
ii) analyzing vectors for two or more expressed genes to determine a co-differential expression probability; and
iii) determining whether said probability for said two or more expressed genes is less than a specified probability threshold.

4. The method of claim 1, further comprising the step of translating said subset of genes of unknown function to generate corresponding polypeptides.

5. A method for analyzing gene expression, the method comprising:
a) receiving a plurality of single channel DNA microarray images;
b) analyzing said images to determine whether elements in said images exceed a signal level threshold;
c) generating an expression data vector for said elements in said images by categorizing whether said elements have a specific signal or a nonspecific signal;
d) analyzing said vectors to determine a co-expression probability; and
e) determining whether said probability is less than a specified probability threshold.

6. The method of claim 5, wherein at least some of said elements in said DNA microarray images correspond to genes of unknown function.

7. The method of claim 5, wherein at least some of said elements in said DNA microarray images correspond to genes of known function.

8. The method of claim 5, wherein said signal level threshold is defined by estimating a distribution of signal values by using negative controls on said microarray.

9. A polynucleotide identified by the method of claim 1.

10. A polypeptide identified by the method of claim 4.

11. A computer program product comprising a machine readable medium on which is provided program instructions for analyzing gene expression, the instructions comprising:
code for receiving a plurality of dual channel DNA microarray images;
code for analyzing said images to determine expression patterns of one or more disease-specific genes and one or more genes of unknown function; and
code for comparing the expression patterns of said disease-specific genes with the expression patterns of the genes of unknown function to identify a subset of the genes of unknown function which have similar expression patterns to those of the disease-specific genes.

12. The computer program product of claim 11, wherein said code for comparing expression patterns comprises
code for generating an expression data vector for each expressed gene by categorizing whether each gene is differential expressed or not differentially expressed;
code for analyzing vectors for two or more expressed genes to determine a co-differential expression probability; and
code for determining whether the probability for said two or more expressed genes is less than a specified probability threshold.

13. The computer program product of claim 11, further comprising code for translating said subset of genes of unknown function to generate corresponding polypeptides.

14. The computer program product of claim 11, wherein said code for obtaining dual channel DNA microarray images comprises
code for receiving a plurality of single channel DNA microarray images; and
code for determining the ratio between said single channel DNA microarray images to yield a plurality of dual channel DNA microarray images.

15. A computing device comprising a memory device configured to store at least temporarily program instructions for analyzing gene expression, the instructions comprising:
code for receiving a plurality of dual channel DNA microarray images;
code for analyzing said images to determine expression patterns of one or more disease-specific genes and one or more genes of unknown function; and
code for comparing the expression patterns of said disease-specific genes with the expression patterns of the genes of unknown function to identify a subset of the genes of unknown function which have similar expression patterns to those of the disease-specific genes.

16. The computing device of claim 15, wherein said code for comparing expression patterns comprises
code for generating an expression data vector for each expressed gene by categorizing whether each gene is differential expressed or not differentially expressed;
code for analyzing vectors for two or more expressed genes to determine a co-differential expression probability; and
code for determining whether the probability for said two or more expressed genes is less than a specified probability threshold.

17. The computing device of claim 15, further comprising code for translating said subset of genes of unknown function to generate corresponding polypeptides.

18. The computing device of claim 15, wherein said code for obtaining dual channel DNA microarray images comprises
code for receiving a plurality of single channel DNA microarray images; and
code for determining the ratio between said single channel DNA microarray images to yield a plurality of dual channel DNA microarray images.

19. The computing device of claim 15, wherein said code for obtaining dual channel DNA microarray images comprises
code for receiving a plurality of single channel DNA microarray images; and
code for determining the ratio between said single channel DNA microarray images to yield a plurality of dual channel DNA microarray images.

20. A substantially purified biomolecule for use in the diagnosis or treatment of a disease associated with cell proliferation, said biomolecule selected from the group consisting of:
(A) a polynucleotide selected from the group consisting of SEQ ID NO: 7, SEQ ID NO:13, and SEQ ID NO:17;
(B) a polynucleotide which encodes a polypeptide selected from the group consisting of SEQ ID NO:8, SEQ ID NO:14, and SEQ ID NO:18;
(C) a polynucleotide having at least 70% identity to the polynucleotide of (A) or (B);
(D) a polynucleotide which is complementary to the polynucleotide of (A), (B), or (C);
(E) a polynucleotide comprising at least 18 sequential nucleotides of the polynucleotide of (A), (B), (C), or (D);
(F) a polypeptide selected from the group consisting of SEQ ID NO:8, SEQ ID NO:14, and SEQ ID NO:18;
(G) a polypeptide having at least 85% identity to the polypeptide of (F); and
(H) a polypeptide comprising at least 6 sequential amino acids of the polypeptide of (F) or (G).

21. The substantially purified biomolecule of claim 20, comprising a polynucleotide sequence selected from the group consisting of:
(A) a polynucleotide selected from the group consisting of SEQ ID NO: 7, SEQ ID NO:13, and SEQ ID NO:17;
(B) a polynucleotide which encodes a polypeptide selected from the group consisting of SEQ ID NO:8, SEQ ID NO:14, and SEQ ID NO:18;
(C) a polynucleotide having at least 70% identity to the polynucleotide of (A) or (B);
(D) a polynucleotide which is complementary to the polynucleotide of (A), (B), or (C);
(E) a polynucleotide comprising at least 18 sequential nucleotides of the polynucleotide of (A), (B), (C), or (D); and
(F) a polynucleotide which hybridizes under stringent conditions to the polynucleotide of (A), (B), (C), (D), or (E).

22. The substantially purified biomolecule of claim 20, comprising a polypeptide sequence selected from the group consisting of:
(A) a polypeptide selected from the group consisting of SEQ ID NO:8, SEQ ID NO:14, and SEQ ID NO:18;
(B) a polypeptide having at least 85% identity to the polypeptide of (A); and
(C) a polypeptide comprising at least 6 sequential amino acids of the polypeptide of (A) or (B).

23. An expression vector comprising the polynucleotide of claim 21.

24. A host cell comprising the expression vector of claim 23.

25. A method for producing a polypeptide of claim 22, the method comprising the steps of:
a) culturing the host cell of claim 24 under conditions suitable for the expression of the polypeptide; and
b) recovering the polypeptide from the host cell culture.

26. A pharmaceutical composition comprising the biomolecule of claim 20 in conjunction with a suitable pharmaceutical carrier.

27. An antibody which specifically binds to the polypeptide of claim 22.
